(19) United States
(12) Patent Application Publication
(10) Pub. No.: US 2024/0261400 A1
(43) Pub. Date: Aug. 8, 2024

(54) METHODS FOR REDUCING OR ELIMINATING THE NEED FOR LIPOPROTEIN APHERESIS IN PATIENTS WITH HYPERLIPIDEMIA BY ADMINISTERING ALIROCUMAB

(71) Applicant: Regeneron Pharmaceuticals, Inc., Tarrytown, NY (US)

(72) Inventors: Robert C. Pordy, Ardsley, NY (US); Garen Manvelian, White Plains, NY (US)

(21) Appl. No.: 18/407,331

(22) Filed: Jan. 8, 2024

Related U.S. Application Data
(63) Continuation of application No. 16/991,269, filed on Aug. 12, 2020, now Pat. No. 11,904,017.
Application No. 16/991,269 is a continuation of application No. 15/238,890, filed on Aug. 17, 2016, now Pat. No. 10,772,956.
(60) Provisional application No. 62/367,374, filed on Jul. 27, 2016.
(60) Provisional application No. 62/311,455, filed on Mar. 22, 2016.
(60) Provisional application No. 62/291,571, filed on Feb. 5, 2016.
(60) Provisional application No. 62/270,790, filed on Dec. 22, 2015.
(60) Provisional application No. 62/264,361, filed on Dec. 8, 2015.
(60) Provisional application No. 62/206,326, filed on Aug. 18, 2015.

(51) Int. Cl. A61K 39/395; A61K 31/397; A61K 31/455; A61K 35/413; A61K 39/00; A61M 1/34; C07K 16/40

(52) U.S. Cl. CPC A61K 39/3955; A61K 31/397; A61K 31/455; A61K 35/413; A61K 39/395; A61M 1/3496; C07K 16/40; A61K 2039/505; A61K 2039/54; A61K 2039/545; A61M 1/34; A61M 2205/33; C07K 2317/21; C07K 2317/56; C07K 2317/565; C07K 2317/76

(57) ABSTRACT

The present invention provides methods for reducing or eliminating a patient's need for lipoprotein apheresis therapy. The methods of the present invention comprise administering to a patient a pharmaceutical composition comprising a PCSK9 inhibitor. In certain embodiments, the PCSK9 inhibitor is an anti-PCSK9 antibody. The methods of the present invention are useful for treating patients with hyperlipidemia and related conditions who are currently being treated with a therapeutic regimen comprising lipoprotein apheresis (e.g., LDL apheresis or Lp(a) apheresis).

This application is a continuation of U.S. patent application Ser. No. 16/991,269, filed Aug. 12, 2020, which is a continuation of U.S. patent application Ser. No. 15/238,890, filed Aug. 17, 2016, now U.S. Pat. No. 10,772,956, which claims the benefit of U.S. Provisional Patent Application Serial Nos. 62/206,326, filed Aug. 18, 2015; 62/264,361, filed Dec. 8, 2015; 62/270,790, filed Dec. 22, 2015; 62/291,571, filed Feb. 5, 2016; 62/311,455, filed Mar. 22, 2016; and 62/367,374, filed Jul. 27, 2016. The disclosures of the aforementioned patent applications are herein incorporated by reference in their entireties.

### FIELD OF THE INVENTION

### Sequence Listing

The instant application contains a Sequence Listing which has been submitted electronically in XML format and is hereby incorporated by reference in its entirety. Said XML file, created on Jan. 5, 2024, is named 749072_SA9-290CON2_ST26.xml and is 229,340 bytes in size.

### FIELD OF THE INVENTION

The present invention relates to the field of therapeutic treatments of diseases and disorders which are associated with elevated levels of lipids and lipoproteins. More specifically, the invention relates to the use of PCSK9 inhibitors to treat patients with hyperlipidemia and related conditions who are currently being treated with a therapeutic regimen comprising lipoprotein apheresis (e.g., LDL apheresis or Lp(a) apheresis).

### BACKGROUND

Hyperlipidemia is a general term that encompasses diseases and disorders characterized by or associated with elevated levels of lipids and/or lipoproteins in the blood. Hyperlipidemias include hypercholesterolemia, hypertriglyceridemia, combined hyperlipidemia, and elevated lipoprotein a (Lp(a)). A particular prevalent form of hyperlipidemia in many populations is hypercholesterolemia.

Hypercholesterolemia, particularly an increase in low-density lipoprotein (LDL) cholesterol (LDL-C) levels, constitutes a major risk for the development of atherosclerosis and coronary heart disease (CHD) (Sharrett et al., 2001, Circulation 104:1108-1113). Low-density lipoprotein cholesterol is identified as the primary target of cholesterol lowering therapy and is accepted as a valid surrogate therapeutic endpoint. Numerous studies have demonstrated that reducing LDL-C levels reduces the risk of CHD with a strong direct relationship between LDL-C levels and CHD events; for each 1 mmol/L (˜40 mg/dL) reduction in LDL-C, cardiovascular disease (CVD) mortality and morbidity is lowered by 22%. Greater reductions in LDL-C produce greater reduction in events, and comparative data of intensive versus standard statin treatment suggest that the lower the LDL-C level, the greater the benefit in patients at very high cardiovascular (CV) risk.

Familial hypercholesterolemia (FH) is an inherited disorder of lipid metabolism that predisposes a person to premature severe cardiovascular disease (CVD). Defects in at least 3 different genes that code for proteins involved in hepatic clearance of low-density lipoprotein (LDL) cholesterol (LDL-C) can cause FH. Examples of such defects include mutations in the gene coding for the LDL receptor (LDLR) that removes LDL-C from the circulation, and in the gene for apolipoprotein (Apo) B, which is the major protein of the LDL particle. In certain cases of FH, the gene coding for proprotein convertase subtilisin/kexin type 9 (PCSK9), an enzyme involved in degrading the LDLR (gain of function mutation), is mutated. In all cases, FH is characterized by an accumulation of LDL-C in the plasma from birth and subsequent development of tendon xanthomas, xanthelasmas, atheromata, and CVD. FH can be classified as either heterozygous FH (heFH) or homozygous FH (hoFH) depending on whether the individual has a genetic defect in one (heterozygous) or both (homozygous) copies of the implicated gene.

Current LDL-C-lowering medications include statins, cholesterol absorption inhibitors, fibrates, niacin, and bile acid sequestrants. Statins are a commonly prescribed treatment for LDL-C lowering. However, despite the availability of such lipid-lowering therapies, many high-risk patients fail to reach their guideline target LDL-C level (Gitt et al., 2010, Clin Res Cardiol 99(11):723-733). For patients who are still unable to achieve guideline target level for LDL-C, despite available lipid-modifying therapy (LMT), mechanical removal of LDL-C by lipoprotein apheresis (e.g., LDL apheresis) is sometimes prescribed. Lipoprotein apheresis removes apoproteinB100-containing lipoproteins from the blood. It is generally regarded as a last-resort option for patients with progressive cardiovascular disease and persistently elevated LDL-C.

However, LDL apheresis is a costly procedure that is invasive and burdensome for patients. Apheresis, in general, involves the mechanical removal blood from a patient; the blood is subjected to centrifugation, filtration or other separation steps outside the body to remove unwanted constituents and then reintroduced back into the patient. Lipoprotein apheresis acutely lowers the LDL-C concentration by 50-75%, which translates to a time-averaged LDL-C reduction of approximately 30% between apheresis procedures. Typical apheresis processes are characterized by a transient reduction in serum lipoprotein concentration that is followed over time by an almost linear return of lipoprotein levels to the elevated “baseline” level. This oscillating pattern of lipoprotein levels, which is characteristic of lipoprotein apheresis therapies, explains the need for periodic apheresis treatments that are required throughout the lifetime of an individual. Furthermore, because of the sparsity of apheresis centers in many geographical locations, many patients must travel a significant distance for this procedure, which is administered over 3 hours and is typically given every week to every 4 weeks, depending on the patient's LDL-C level and cardiovascular risk. In addition, this procedure may require placement of a shunt for frequent vascular access. Low-density lipoprotein apheresis is generally well tolerated, but may result in hypotension, hypocalcemia, allergic reactions, and an acute decrease in serum protein levels. It has been documented that quality of life (QoL, as determined by questionnaire) was lower in patients undergoing apheresis in addition to lipid-lowering drugs compared to patients treated only with lipid-lowering drugs (Schiel et al., 1995, Int J Artif Organs 18:786-793). Thus, patients who are not at LDL-C goal despite receiving an optimized LMT regimen, and who require apheresis to lower LDL-C, would greatly benefit from alternative LDL-C lowering therapies that are capable of reducing or eliminating the need for apheresis.

### BRIEF SUMMARY OF THE INVENTION

The present invention provides methods for treating hyperlipidemia in patients receiving lipoprotein apheresis therapy. The therapeutic methods of the present invention result in a lowering of serum lipoprotein levels and thereby serve to eliminate or reduce a patient's need for lipoprotein apheresis. In certain embodiments, the frequency of apheresis treatments required by a patient to achieve a target lipoprotein level (e.g., a target LDL-C level) is reduced by application of the therapeutic methods of the present invention. In certain embodiments, a patient's need for apheresis to achieve a target lipoprotein level (e.g., a target LDL-C level) is eliminated by application of the therapeutic methods of the present invention.

According to one aspect, the methods of the present invention comprise administering one or more doses of a PCSK9 inhibitor to a patient who is being or has been treated (e.g., within the last six months) with lipoprotein apheresis, wherein administration of the PCSK9 inhibitor to the patient results in a lowering of the level of at least one lipoprotein in the serum of the patient and consequently reduces or eliminates the need for lipoprotein apheresis therapy by the patient.

According to another aspect, the methods of the present invention comprise selecting a patient with hypercholesterolemia who is being or has been treated with lipoprotein apheresis at an initial (pre-treatment) frequency, and administering one or more doses of a PCSK9 inhibitor to the patient, thereby lowering the level of at least one lipoprotein in the serum of the patient and consequently reducing frequency of lipoprotein apheresis required by the patient to achieve a target lipoprotein level.

Patients who are treated or treatable by the methods of the present invention include, e.g., patients with hypercholesterolemia, including patients with familial hypercholesterolemia (FH). In certain embodiments, the patients who are treated or treatable by the methods of the present invention are patients who are diagnosed with (or otherwise known to have) homozygous FH (hoFH) or heterozygous FH (heFH).

According to certain embodiments of the present invention, the PCSK9 inhibitor is administered to the patient as an add-on therapy to the patient's existing lipid-lowering therapy (e.g., on top of the patient's background statin therapy).

The present invention also provides pharmaceutical compositions comprising a PCSK9 inhibitor for use in reducing or eliminating the need for lipoprotein apheresis therapy, or for use in reducing the frequency of lipoprotein apheresis therapy by a patient.

Exemplary PCSK9 inhibitors that may be used in the context of the methods of the present invention include, e.g., anti-PCSK9 antibodies, small molecule PCSK9 inhibitors, and scaffold-based PCSK9-binding molecules.

Other embodiments of the present invention will become apparent from a review of the ensuing detailed description.

### BRIEF DESCRIPTION OF THE FIGURES

FIG. 1 is a diagram illustrating the overall design of the study described in Example 2 herein. The study included a 2 week screening period, an 18 week double-blind treatment period, and an optional 8 week follow-up/open-label extension period.

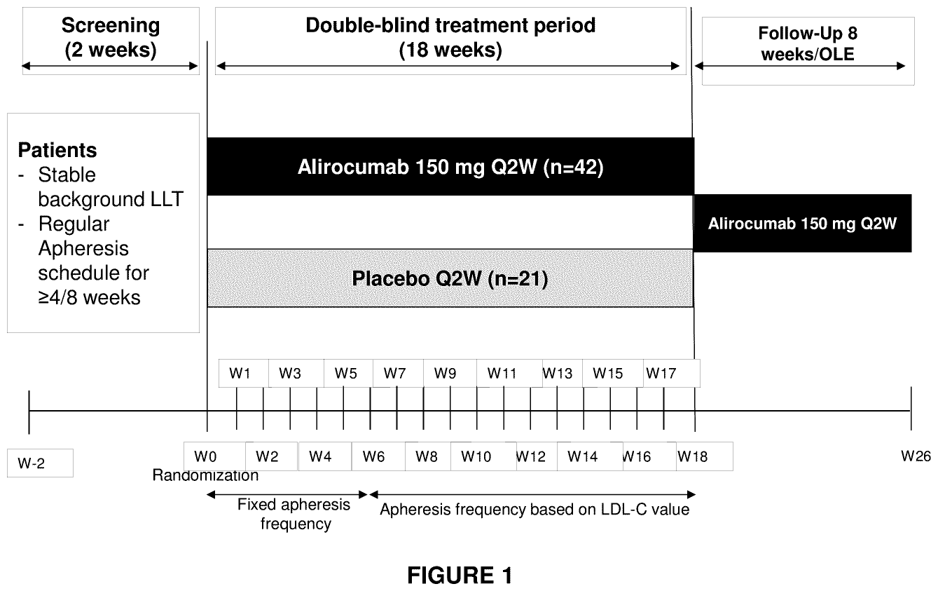

FIG. 2 is a waterfall plot showing individual patients' reductions in rate of apheresis treatments from week 7 to week 18 in the placebo and alirocumab-treated patient groups (ITT population). Only apheresis treatment skipping per point-of-care LDL-C value is counted as “apheresis not occurred”. Missing apheresis treatment information (for any reason) from week 7 to week 18 is imputed to an outcome of the apheresis treatment “occurred” at the visit.

FIG. 3 shows the standardized apheresis rate from week 7 to week 18 in the placebo and alirocumab-treated patient groups. Apheresis rates are shown along the x-axis; the y-axis shows the percentage of patients exhibiting the corresponding apheresis rate range.

FIG. 4 shows the percent of patients receiving apheresis in the placebo and alirocumab-treated patient groups at different time points from week 7 through week 18 of the study. The patients shown in this chart are those who were receiving apheresis weekly (QW) at the start of the study.

FIG. 5 shows the percent of patients receiving apheresis in the placebo and alirocumab-treated patient groups at different time points from week 7 through week 18 of the study. The patients shown in this chart are those who were receiving apheresis once every other week (Q2W) at the start of the study.

FIG. 6 shows the percent of patients receiving apheresis in the placebo and alirocumab-treated patient groups at different time points from week 7 through week 18 of the study. The patients shown in this chart represent all patients enrolled in the study.

FIG. 7 shows the calculated LDL-C percentage change from baseline in the placebo and alirocumab-treated patient groups at different time points throughout the course of the study. The black arrow labeled “apheresis varies” indicates the time point at which the apheresis frequency going forward was determined for individual patients based on LDL-C level. Prior to this time point, patients received apheresis at a fixed frequency based on the individual patient's established schedule.

FIG. 8 shows the calculated LDL-C levels (expressed in terms of LS-mean [+/−SE] mg/dL) in the placebo and alirocumab-treated patient groups at different time points throughout the course of the study. The black arrow labeled “apheresis varies” indicates the time point at which the apheresis frequency going forward was determined for individual patients based on LDL-C level. Prior to this time point, patients received apheresis at a fixed frequency based on the individual patient's established schedule.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present invention, the preferred methods and materials are now described. All publications mentioned herein are incorporated herein by reference to describe in their entirety.

### Methods for Reducing or Eliminating the Need for Lipoprotein Apheresis

The present invention relates generally to methods and compositions for reducing lipoprotein levels in patients who are undergoing or have undergone (e.g., within the last six months or more) lipoprotein apheresis to lower serum lipoprotein levels (e.g., LDL-C and/or Lp(a)). According to certain embodiments, the methods of the present invention result in a reduction in lipoprotein levels in the serum of such patients so that the need for lipoprotein apheresis is reduced or eliminated.

As used herein, the term “lipoprotein” means a biomolecular particle containing both protein and lipid. Examples of lipoproteins include, e.g., low density lipoprotein (LDL), very low density lipoprotein (VLDL), intermediate density lipoprotein (IDL), and lipoprotein (a) (Lp(a)).

As used in the context of the present invention, “lipoprotein apheresis” means a therapeutic process involving the mechanical removal blood from a patient, followed by the removal of lipoprotein (e.g., LDL-C and/or Lp(a)) from the patient's blood by processes such as filtration, adsorption, precipitation, and the like, and finally reintroduction of the treated blood back into the patient's blood stream. For purposes of the present disclosure, “LDL apheresis” and “Lp(a) apheresis” are considered types of apheresis, and therefore are encompassed by the definition of the more general definition “lipoprotein apheresis.”

Specific types of lipoprotein apheresis that are included in the context of the present invention include, e.g., double membrane filtration, immunoadsorption, heparin-induced LDL precipitation, direct adsorption of lipids, dextran sulfate-cellulose adsorption (plasma or whole blood), heparin extracorporeal LDL precipitation (HELP) system, DFPP and thermofiltration plasmapheresis, and hemoperfusion.

The present invention, according to certain embodiments, includes methods for eliminating the need for lipoprotein apheresis therapy. As used herein, a particular patient's “need for lipoprotein apheresis therapy” is determined by a physician, physician's assistant, diagnostician, or other medical professional on the basis of the level of one or more lipoproteins (e.g., LDL-C and/or Lp(a)) measured or otherwise detected in the serum of the patient. A patient's “need for lipoprotein apheresis therapy” may also be determined or influenced by other factors such as the patient's family history, medical background, current therapeutic treatment status, as well as generally accepted or prevailing lipoprotein targets adopted by national medical associations and physicians' groups. For example, in certain contexts, an LDL-C level of greater than or equal to about 70 mg/dL indicates a “need for lipoprotein apheresis therapy” by a patient. In other contexts, an LDL-C level of greater than or equal to about 100 mg/dl indicates a “need for lipoprotein apheresis therapy” by a patient. In certain contexts, an LDL-C level greater than or equal to about 150 mg/dl, 200 mg/dL, 250 mg/dL, 300 mg/dL, 400 mg/dL or higher, indicates a “need for lipoprotein apheresis therapy” by a patient. In yet other contexts, whether or not a particular percentage reduction in LDL-C or Lp(a) level is met, relative to the patient's LDL-C or Lp(a) level at a particular start point (“baseline”) can be used to determine whether the patient has a “need for lipoprotein apheresis therapy.” For instance, a reduction in LDL-C or Lp(a) of less than 50% (e.g., less than 40%, less than 35%, less than 30%, less than 25%, etc.) from baseline may signify a “need for lipoprotein apheresis therapy.”

The present invention, according to certain embodiments, includes methods for reducing the frequency of lipoprotein apheresis therapy in a patient. As will be appreciated by persons of ordinary skill in the art, a patient may be treated with lipoprotein apheresis at a particular frequency in order to achieve a particular lipoprotein goal (e.g., an LDL-C level of less than 100 mg/dl, an LDL-C level of less than 70 mg/dl, etc.). The prescribed frequency is determined based on the number of apheresis treatments required by a patient during a particular time period (e.g., weekly, monthly, etc.) in order to achieve and maintain the lipoprotein goal for the patient. Exemplary lipoprotein apheresis frequencies include, e.g., once a week, once every two weeks, once every 3 weeks, once every four weeks, once every five weeks, once a month, twice a month, once every two months, etc. The present invention includes methods for reducing the frequency of lipoprotein apheresis therapy in a patient by administering to the patient one or more doses of a PCSK9 inhibitor. According to certain embodiments of the present invention, the frequency of apheresis following administration of one or more doses of a PCSK9 inhibitor is reduced by at least 50% from the patient's pre-treatment apheresis frequency. For example, if a patient, prior to treatment with a PCSK9 inhibitor, is on a lipoprotein apheresis treatment regimen at a frequency of once a week (QW), and after treatment with a PCSK9 inhibitor the apheresis frequency for the patient is reduced to once every 2 weeks (Q2W), then the patient is said to have achieved a 50% reduction in lipoprotein apheresis frequency following treatment. In certain embodiments, the frequency of apheresis following administration of one or more doses of a PCSK9 inhibitor according to the present invention, is reduced by 75%, or by 100% (i.e., elimination of the need for lipoprotein apheresis following treatment).

Lipoprotein apheresis frequency, in the context of the present invention, can be expressed in terms of a pre-treatment frequency and a post-treatment frequency. “Pre-treatment frequency” means the frequency of apheresis therapy required by a patient in order to achieve and/or maintain a particular target lipoprotein level prior to commencement of a therapeutic regimen comprising administration of a PCSK9 inhibitor. “Post-treatment frequency” means the frequency of apheresis therapy required by a patient in order to achieve and/or maintain a particular target lipoprotein level after to commencement of a therapeutic regimen comprising administration of a PCSK9 inhibitor. The frequency of apheresis therapy required to achieve and/or maintain a particular target lipoprotein level for a particular patient is preferably determined by a qualified medical professional based on generally accepted criteria in the art, including the serum level of lipoprotein sought to be reduced or otherwise controlled.

The present invention, accordingly, includes methods of treatment comprising administration of one or more doses of a PCSK9 inhibitor to a patient, whereby the patient's post-treatment frequency of lipoprotein apheresis is less than the patient's pre-treatment frequency of lipoprotein apheresis. For example, the present invention includes therapeutic methods comprising administering one or more doses of a PCSK9 inhibitor to a patient who is undergoing or has undergone lipoprotein apheresis therapy at a pre-treatment frequency of, e.g., once a week, wherein, after receiving one or more doses of the PCSK9 inhibitor, the frequency of lipoprotein apheresis required by the patient to achieve and/or maintain a particular target lipoprotein level (i.e., post-treatment frequency) is, e.g., once every two weeks, once every three weeks, once every four weeks, or less frequently. In some instances, the need for lipoprotein apheresis required by the patient to achieve and/or maintain a particular target lipoprotein level is eliminated altogether following administration of one or more doses of the PCSK9 inhibitor.

According to certain embodiments, the present invention comprises methods for reducing or eliminating the need for lipoprotein apheresis therapy, wherein the methods comprise selecting a patient with hyperlipidemia (e.g., hypercholesterolemia) who has been treated with lipoprotein apheresis within the last month, the last 2 months, the last 3 months, the last 4 months, the last 5 months, the last 6 months, or for a longer period, and administering one or more doses of a PCSK9 inhibitor to the patient. The methods according to this aspect of the invention result in lowering the level of at least one lipoprotein in the serum of the patient, and consequently allow for a reduction or elimination of the need for lipoprotein apheresis therapy by the patient. For example, in certain embodiments of the present invention, following administration of one or more doses of a PCSK9 inhibitor, the serum LDL-C level of the patient is reduced to less than a defined level (e.g., less than 100 mg/dL or less than 70 mg/dL), such that the post-treatment frequency of lipoprotein apheresis therapy required by the patient to achieve and/or maintain a particular target lipoprotein level is reduced, or it is concluded that lipoprotein apheresis is no longer required.

In certain embodiments, rate (or frequency) of apheresis is expressed as the normalized rate of apheresis required by a patient to achieve and/or maintain a particular target lipoprotein level. As used herein, the normalized rate of apheresis for a particular patient is defined as the number of actual apheresis treatments received by the patient over defined period of time following initiation of an anti-PCSK9 therapeutic regimen, divided by the number of apheresis treatments received by the patient over an equivalent period of time prior to initiation of the anti-PCSK9 therapeutic regimen. For example, if prior to initiating a therapeutic regiment comprising administration of an anti-PCSK9 antibody, a patient received 8 apheresis treatments over an 8 week period (e.g., once a week), and after initiation of the anti-PCSK9 therapeutic regiment the patient received 2 apheresis treatments over an 8 week period (e.g., once every four weeks), then the patient's normalized rate of apheresis is 2/8=0.25. The present invention includes methods by which the normalized rate of apheresis for a patient following administration of a PCSK9 inhibitor is, e.g., less than 0.9, less than 0.8, less than 0.7, less than 0.6, less than 0.5, less than 0.4, less than 0.3, less than 0.2, or less than 0.1.

According to certain embodiments, the patient who is treatable by the methods of the present invention has hypercholesterolemia (e.g., a serum LDL-C concentration of greater than or equal to 70 mg/dL, or a serum LDL-C concentration greater than or equal to 100 mg/dL). In certain embodiments, the patient's hypercholesterolemia is inadequately controlled by statin therapy. For example, the present invention includes methods for reducing or eliminating the frequency of lipoprotein apheresis therapy by a patient who has hypercholesterolemia that is inadequately controlled by a daily dose of a statin selected from the group consisting of atorvastatin (including atorvastatin+ezetimibe), rosuvastatin, cerivastatin, pitavastatin, fluvastatin, lovastatin, simvastatin (including simvastatin+ezetimibe), pravastatin, and combinations thereof. The present invention also includes methods for reducing or eliminating the frequency of lipoprotein apheresis therapy by a patient who has hypercholesterolemia and who exhibits statin intolerance or who otherwise experiences adverse or undesirable reaction(s) to statin therapy (e.g., skeletal muscle pain, aches, weakness or cramping [e.g., myalgia, myopathy, rhabdomyolysis, etc.]).

### Methods for Treating Lipid Lesions

The present invention further relates to methods and compositions for treating, reversing or resolving physical manifestations of hypercholesterolemia. According to certain embodiments, the present invention provides methods for treating a patient having a lipid lesion associated with hypercholesterolemia. For example, the present invention provides method for treating a patient having one or more xanthelasmata. The methods and compositions according to this aspect of the invention comprise administering one or more doses of PCSK9 inhibitor to a patient in need thereof, wherein lipid lesions that were existent in the patient prior to treatment with a PCSK9 inhibitor are reduced, resolved or eliminated following administration of one or more doses of the PCSK9 inhibitor to the patient. According to certain embodiments, the present invention comprises methods for treating a patient having a lipid lesion associated with hypercholesterolemia, wherein the methods comprise selecting a patient with a lipid lesion (e.g., a xanthelasma), and administering one or more doses of a PCSK9 inhibitor to the patient.

### Patient Selection

The present invention includes methods and composition useful for treating patients who are undergoing, or have recently undergone, lipoprotein apheresis (e.g., within the last six months, within the last 12 weeks, within the last 8 weeks, within the last 6 weeks, within the last 4 weeks, within the last 2 weeks, etc.). The patients who are treatable by the methods of the present invention may also exhibit one or more of additional selection criteria. For example, a patient may be selected for treatment with the methods of the present invention if the patient is diagnosed with or identified as being at risk of developing a hypercholesterolemia condition such as, e.g., heterozygous Familial Hypercholesterolemia (heFH), homozygous Familial Hypercholesterolemia (hoFH), Autosomal Dominant Hypercholesterolemia (ADH, e.g., ADH associated with one or more gain-of-function mutations in the PCSK9 gene), autosomal recessive hypercholesterolemia (ARH, e.g., ARH associated with mutations in LDLRAP1), as well as incidences of hypercholesterolemia that are distinct from Familial Hypercholesterolemia (nonFH). Diagnosis of familial hypercholesterolemia (e.g., heFH or hoFH) can be made by genotyping and/or clinical criteria. For patients who are not genotyped, clinical diagnosis may be based on either the Simon Broome criteria with a criteria for definite FH, or the WHO/Dutch Lipid Network criteria with a score >8 points.

According to certain embodiments, the patient may be selected on the basis of having a history of coronary heart disease (CHD). As used herein a “history of CHD” (or “documented history of CHD”) includes one or more of: (i) acute myocardial infarction (MI); (ii) silent MI; (iii) unstable angina; (iv) coronary revascularization procedure (e.g., percutaneous coronary intervention [PCI] or coronary artery bypass graft surgery [CABG]); and/or (v) clinically significant CHD diagnosed by invasive or non-invasive testing (such as coronary angiography, stress test using treadmill, stress echocardiography or nuclear imaging).

According to certain embodiments, the patient may be selected on the basis of having non-coronary heart disease cardiovascular disease (“non-CHD CVD”). As used herein, “non-CHD CVD” includes one or more of: (i) documented previous ischemic stroke with a focal ischemic neurological deficit that persisted more than 24 hours, considered as being of atherothrombotic origin; (ii) peripheral arterial disease; (iii) abdominal aortic aneurysm; (iv) atherosclerotic renal artery stenosis; and/or (v) carotid artery disease (transient ischemic attacks or >50% obstruction of a carotid artery).

According to certain embodiments, the patient may be selected on the basis of having one or more additional risk factors such as, e.g., (i) documented moderate chronic kidney disease (CKD) as defined by 30≤eGFR<60 mL/min/1.73 m2 for 3 months or more; (ii) type 1 or type 2 diabetes mellitus with or without target organ damage (e.g., retinopathy, nephropathy, microalbuminuria); (iii) a calculated 10-year fatal CVD risk SCORE≥5% (ESC/EAS Guidelines for the management of dyslipidemias, Conroy et al., 2003, Eur. Heart J. 24:987-1003).

According to certain embodiments, the patient may be selected on the basis of having one or more additional risk factors selected from the group consisting of age (e.g., older than 40, 45, 50, 55, 60, 65, 70, 75, or 80 years), race, national origin, gender (male or female), exercise habits (e.g., regular exerciser, non-exerciser), other preexisting medical conditions (e.g., type-II diabetes, high blood pressure, etc.), and current medication status (e.g., currently taking beta blockers, niacin, ezetimibe, fibrates, omega-3 fatty acids, bile acid resins, etc.).

According to the present invention, patients may be selected on the basis of a combination of one or more of the foregoing selection criteria or therapeutic characteristics. For example, according to certain embodiments, a patient suitable for treatment with the methods of the present invention, in addition to undergoing, or have recently undergone (e.g., within the last six months), lipoprotein apheresis, may further be selected on the basis of having heFH or non-FH in combination with: (i) a history of documented CHD, (ii) non-CHD CVD, and/or (iii) diabetes mellitus with target organ damage; such patients may also be selected on the basis of having a serum LDL-C concentration of greater than or equal to 70 mg/dL.

According to certain other embodiments, a patient suitable for treatment with the methods of the present invention, in addition to having hypercholesterolemia that is not adequately controlled by a daily moderate-dose therapeutic statin regimen, may further be selected on the basis of having heFH or non-FH without CHD, or non-CHD CVD, but having either (i) a calculated 10-year fatal CVD risk SCORE≥5%; or (ii) diabetes mellitus without target organ damage; such patients may also be selected on the basis of having a serum LDL-C concentration of greater than or equal to 100 mg/dL.

### Administration of a PCSK9 Inhibitor as Add-on Therapy

The present invention includes methods of treatment wherein a patient who is undergoing, or has recently undergone, lipoprotein apheresis is administered a PCSK9 inhibitor according to a particular dosing amount and frequency, and wherein the PCSK9 inhibitor is administered as an add-on to the patient's pre-existing lipid lowering therapy (if applicable), such as an add-on to the patient's pre-existing daily therapeutic statin regimen.

For example, the methods of the present invention include add-on therapeutic regimens wherein the PCSK9 inhibitor is administered as add-on therapy to the same stable daily therapeutic statin regimen (i.e., same dosing amount of statin) that the patient was on prior to receiving the PCSK9 inhibitor. In other embodiments, the PCSK9 inhibitor is administered as add-on therapy to a therapeutic statin regimen comprising a statin in an amount that is more than or less than the dose of stain the patient was on prior to receiving the PCSK9 inhibitor. For example, after starting a therapeutic regimen comprising a PCSK9 inhibitor administered at a particular dosing frequency and amount, the daily dose of statin administered or prescribed to the patient may (a) stay the same, (b) increase, or (c) decrease (e.g., up-titrate or down-titrate) in comparison to the daily statin dose the patient was taking before starting the PCSK9 inhibitor therapeutic regimen, depending on the therapeutic needs of the patient.

### Therapeutic Efficacy

The methods of the present invention result in the reduction in serum levels of one or more lipid component selected from the group consisting of LDL-C, ApoB100, non-HDL-C, total cholesterol, VLDL-C, triglycerides, Lp(a) and remnant cholesterol. The lipoprotein-lowering effects of the therapeutic regimens of the present invention consequently lead to a reduced frequency, or eliminated need for, lipoprotein apheresis in order to achieve a target lipoprotein level by the patient. For example, according to certain embodiments of the present invention, administration of a pharmaceutical composition comprising a PCSK9 inhibitor to a patient who is undergoing, or has recently undergone, lipoprotein apheresis will result in a mean percent reduction from baseline in serum low density lipoprotein cholesterol (LDL-C) of at least about 25%, 30%, 40%, 50%, 60%, or greater; a mean percent reduction from baseline in ApoB100 of at least about 25%, 30%, 40%, 50%, 60%, or greater; a mean percent reduction from baseline in non-HDL-C of at least about 25%, 30%, 40%, 50%, 60%, or greater; a mean percent reduction from baseline in total cholesterol of at least about 10%, 15%, 20%, 25%, 30%, 35%, or greater; a mean percent reduction from baseline in VLDL-C of at least about 5%, 10%, 15%, 20%, 25%, 30%, or greater; a mean percent reduction from baseline in triglycerides of at least about 5%, 10%, 15%, 20%, 25%, 30%, 35% or greater; and/or a mean percent reduction from baseline in Lp(a) of at least about 5%, 10%, 15%, 20%, 25%, or greater.

The present invention includes methods for treating a patient with hypercholesterolemia, the methods comprising administering multiple doses of an anti-PCSK9 antibody to the patient at a dosing amount of about 75 to 150 mg per dose, and a dosing frequency of about once every two weeks, (or a dosing regimen in accordance with an up-titration dosing regimen as described elsewhere herein), wherein the patient is undergoing, or has recently undergone, lipoprotein apheresis, and wherein, after about 12, 14, 16, 18, 20, 22, 24 or more weeks of treatment with the anti-PCSK9 antibody, the patient exhibits a reduction in LDL-C level from baseline of at least 50%, and therefore results in a reduced frequency or elimination of the need for lipoprotein apheresis by the patient. In certain embodiments, following one or more weeks of treatment with the anti-PCSK9 antibody, the patient exhibits a reduction in LDL-C level from baseline of about 55%, 60%, 70%, or more.

### PCSK9 Inhibitors

The methods of the present invention comprise administering to a patient a therapeutic composition comprising a PCSK9 inhibitor. As used herein, a “PCSK9 inhibitor” is any agent which binds to or interacts with human PCSK9 and inhibits the normal biological function of PCSK9 in vitro or in vivo. Non-limiting examples of categories of PCSK9 inhibitors include small molecule PCSK9 antagonists, nucleic acid-based inhibitors of PCSK9 expression or activity (e.g., siRNA or antisense), peptide-based molecules that specifically interact with PCSK9 (e.g., peptibodies), receptor molecules that specifically interact with PCSK9, proteins comprising a ligand-binding portion of an LDL receptor, PCSK9-binding scaffold molecules (e.g., DARPins, HEAT repeat proteins, ARM repeat proteins, tetratricopeptide repeat proteins, fibronectin-based scaffold constructs, and other scaffolds based on naturally occurring repeat proteins, etc., [see, e.g., Boersma and Pluckthun, 2011, Curr. Opin. Biotechnol. 22:849-857, and references cited therein]), and anti-PCSK9 aptamers or portions thereof. According to certain embodiments, PCSK9 inhibitors that can be used in the context of the present invention are anti-PCSK9 antibodies or antigen-binding fragments of antibodies that specifically bind human PCSK9.

The term “human proprotein convertase subtilisin/kexin type 9” or “human PCSK9” or “hPCSK9”, as used herein, refers to PCSK9 having the nucleic acid sequence shown in SEQ ID NO:197 and the amino acid sequence of SEQ ID NO: 198, or a biologically active fragment thereof.

The term “antibody”, as used herein, is intended to refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-PCSK9 antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-CL; (viii) V_{L}-C_{H}1; (ix) V_{L}-C_{H}2; (x) V_{L}-C_{H}3; (xi) V_{L}-C_{H}1-C_{H}2; (Xii) V_{L}-C_{H}1-C_{H}2-C_{H}3; (xiii) V_{L}-C_{H}2-C_{H}3; and (xiv) V_{L}-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody”, as used herein, is intended to include antibodies having variable and constant regions derived from human germline immunoglobulin sequences. The human antibodies of the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antibodies having one or more mutations in the hinge, C_{H}2 or C_{H}3 region which may be desirable, for example, in production, to improve the yield of the desired antibody form.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PCSK9, as used in the context of the present invention, includes antibodies that bind PCSK9 or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PCSK9, however, have cross-reactivity to other antigens, such as PCSK9 molecules from other (non-human) species.

The anti-PCSK9 antibodies useful for the methods of the present invention may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes methods involving the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present invention.

The present invention also includes methods involving the use of anti-PCSK9 antibodies comprising variants of any of the HCVR, LCVR, and/or CDR amino acid sequences disclosed herein having one or more conservative substitutions. For example, the present invention includes the use of anti-PCSK9 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “surface plasmon resonance”, as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, NJ).

The term “K_{D}”, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

According to certain embodiments, the anti-PCSK9 antibody used in the methods of the present invention is an antibody with pH-dependent binding characteristics. As used herein, the expression “pH-dependent binding” means that the antibody or antigen-binding fragment thereof exhibits “reduced binding to PCSK9 at acidic pH as compared to neutral pH” (for purposes of the present disclosure, both expressions may be used interchangeably). For the example, antibodies “with pH-dependent binding characteristics” includes antibodies and antigen-binding fragments thereof that bind PCSK9 with higher affinity at neutral pH than at acidic pH. In certain embodiments, the antibodies and antigen-binding fragments of the present invention bind PCSK9 with at least 3, 5, 10, 15, 20, 25, 30, 35, 40, 45, 50, 55, 60, 65, 70, 75, 80, 85, 90, 95, 100, or more times higher affinity at neutral pH than at acidic pH.

According to this aspect of the invention, the anti-PCSK9 antibodies with pH-dependent binding characteristics may possess one or more amino acid variations relative to the parental anti-PCSK9 antibody. For example, an anti-PCSK9 antibody with pH-dependent binding characteristics may contain one or more histidine substitutions or insertions, e.g., in one or more CDRs of a parental anti-PCSK9 antibody. Thus, according to certain embodiments of the present invention, methods are provided comprising administering an anti-PCSK9 antibody which comprises CDR amino acid sequences (e.g., heavy and light chain CDRs) which are identical to the CDR amino acid sequences of a parental anti-PCSK9 antibody, except for the substitution of one or more amino acids of one or more CDRs of the parental antibody with a histidine residue. The anti-PCSK9 antibodies with pH-dependent binding may possess, e.g., 1, 2, 3, 4, 5, 6, 7, 8, 9, or more histidine substitutions, either within a single CDR of a parental antibody or distributed throughout multiple (e.g., 2, 3, 4, 5, or 6) CDRs of a parental anti-PCSK9 antibody. For example, the present invention includes the use of anti-PCSK9 antibodies with pH-dependent binding comprising one or more histidine substitutions in HCDR1, one or more histidine substitutions in HCDR2, one or more histidine substitutions in HCDR3, one or more histidine substitutions in LCDR1, one or more histidine substitutions in LCDR2, and/or one or more histidine substitutions in LCDR3, of a parental anti-PCSK9 antibody.

As used herein, the expression “acidic pH” means a pH of 6.0 or less (e.g., less than about 6.0, less than about 5.5, less than about 5.0, etc.). The expression “acidic pH” includes pH values of about 6.0, 5.95, 5.90, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

Non-limiting examples of anti-PCSK9 antibodies that can be used in the context of the present invention include, e.g., alirocumab, evolocumab, bococizumab, lodelcizumab, ralpancizumab, or antigen-binding portions of any of the foregoing antibodies.

### Preparation of Human Antibodies

Methods for generating human antibodies in transgenic mice are known in the art. Any such known methods can be used in the context of the present invention to make human antibodies that specifically bind to human PCSK9.

Using VELOCIMMUNE™ technology (see, for example, U.S. Pat. No. 6,596,541, Regeneron Pharmaceuticals) or any other known method for generating monoclonal antibodies, high affinity chimeric antibodies to PCSK9 are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc, using standard procedures known to those skilled in the art. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

In general, the antibodies that can be used in the methods of the present invention possess high affinities, as described above, when measured by binding to antigen either immobilized on solid phase or in solution phase. The mouse constant regions are replaced with desired human constant regions to generate the fully human antibodies of the invention. While the constant region selected may vary according to specific use, high affinity antigen-binding and target specificity characteristics reside in the variable region.

Specific examples of human antibodies or antigen-binding fragments of antibodies that specifically bind PCSK9 which can be used in the context of the methods of the present invention include any antibody or antigen-binding fragment which comprises the three heavy chain CDRs (HCDR1, HCDR2 and HCDR3) contained within a heavy chain variable region (HCVR) having an amino acid sequence selected from the group consisting of SEQ ID NOs:1 and 11, or a substantially similar sequence thereof having at least 90%, at least 95%, at least 98% or at least 99% sequence identity. Alternatively, specific examples of human antibodies or antigen-binding fragments of antibodies that specifically bind PCSK9 which can be used in the context of the methods of the present invention include any antibody or antigen-binding fragment which comprises the three heavy chain CDRs (HCDR1, HCDR2 and HCDR3) contained within a heavy chain variable region (HCVR) having an amino acid sequence selected from the group consisting of SEQ ID NOs 37, 45, 53, 61, 69, 77, 85, 93, 101, 109, 117, 125, 133, 141, 149, 157, 165, 173, 181, and 189, or a substantially similar sequence thereof having at least 90%, at least 95%, at least 98% or at least 99% sequence identity. The antibody or antigen-binding fragment may comprise the three light chain CDRs (LCVR1, LCVR2, LCVR3) contained within a light chain variable region (LCVR) having an amino acid sequence selected from the group consisting of SEQ ID NOs 6 and 15, or a substantially similar sequence thereof having at least 90%, at least 95%, at least 98% or at least 99% sequence identity. Alternatively, the antibody or antigen-binding fragment may comprise the three light chain CDRs (LCVR1, LCVR2, LCVR3) contained within a light chain variable region (LCVR) having an amino acid sequence selected from the group consisting of SEQ ID NOs 41, 49, 57, 65, 73, 81, 89, 97, 105, 113, 121, 129, 137, 145, 153, 161, 169, 177, 185, and 193, or a substantially similar sequence thereof having at least 90%, at least 95%, at least 98% or at least 99% sequence identity.

Sequence identity between two amino acids sequences is determined over the entire length of the reference amino acid sequence, i.e. the amino acid sequence identified with a SEQ ID NO, using the best sequence alignment and/or over the region of the best sequence alignment between the two amino acid sequences, wherein the best sequence alignment can be obtained with art known tools, e.g. Align, using standard settings, preferably EMBOSS::needle, Matrix: Blosum62, Gap Open 10.0, Gap Extend 0.5.

In certain embodiments of the present invention, the antibody or antigen-binding protein comprises the six CDRs (HCDR1, HCDR2, HCDR3, LCDR1, LCDR2 and LCDR3) from the heavy and light chain variable region amino acid sequence pairs (HCVR/LCVR) selected from the group consisting of SEQ ID NOs:1/6 and 11/15. Alternatively, in certain embodiments of the present invention, the antibody or antigen-binding protein comprises the six CDRs (HCDR1, HCDR2, HCDR3, LCDR1, LCDR2 and LCDR3) from the heavy and light chain variable region amino acid sequence pairs (HCVR/LCVR) selected from the group consisting of SEQ ID NOs:37/41, 45/49, 53/57, 61/65, 69/73, 77/81, 85/89, 93/97, 101/105, 109/113, 117/121, 125/129, 133/137, 141/145, 149/153, 157/161, 165/169, 173/177, 181/185, and 189/193.

In certain embodiments of the present invention, the anti-PCSK9 antibody, or antigen-binding protein, that can be used in the methods of the present invention has HCDR1/HCDR2/HCDR3/LCDR1/LCDR2/LCDR3 amino acid sequences selected from SEQ ID NOs: 2/3/4/7/8/10 (mAb316P [also referred to as “REGN727,” or “alirocumab”]) and 12/13/14/16/17/18 (mAb300N) (See US Patent App. Publ No. 2010/0166768) and 12/13/14/16/17/18, wherein SEQ ID NO:16 comprises a substitution of histidine for leucine at amino acid residue 30 (L30H).

In certain embodiments of the present invention, the antibody or antigen-binding protein comprises HCVR/LCVR amino acid sequence pairs selected from the group consisting of SEQ ID NOs:1/6 and 11/15. In certain exemplary embodiments, the antibody or antigen-binding protein comprises an HCVR amino acid sequence of SEQ ID NO:1 and an LCVR amino acid sequence of SEQ ID NO:6. In certain exemplary embodiments, the antibody or antigen-binding protein comprises an HCVR amino acid sequence of SEQ ID NO:11 and an LCVR amino acid sequence of SEQ ID NO:15. In certain exemplary embodiments, the antibody or antigen-binding protein comprises an HCVR amino acid sequence of SEQ ID NO:11 and an LCVR amino acid sequence of SEQ ID NO:15 comprising a substitution of histidine for leucine at amino acid residue 30 (L30H).

### Pharmaceutical Compositions and Methods of Administration

The present invention includes methods which comprise administering a PCSK9 inhibitor to a patient, wherein the PCSK9 inhibitor is contained within a pharmaceutical composition. The pharmaceutical compositions of the invention are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Exemplary pharmaceutical formulations comprising anti-PCSK9 antibodies that can be used in the context of the present invention include any of the formulations as set forth in U.S. Pat. No. 8,795,669 (describing, inter alia, exemplary formulations comprising alirocumab), or in WO2013/166448, or WO2012/168491.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Dosage

The amount of PCSK9 inhibitor (e.g., anti-PCSK9 antibody) administered to a subject according to the methods of the present invention is, generally, a therapeutically effective amount. As used herein, the phrase “therapeutically effective amount” means a dose of PCSK9 inhibitor that results in a detectable reduction (at least about 5%, 10%, 15%, 20%, 25%, 30%, 35%, 40%, 45%, 50%, 55%, 60%, 65%, 70%, 75%, or more from baseline) in one or more parameters selected from the group consisting of LDL-C, ApoB100, non-HDL-C, total cholesterol, VLDL-C, triglycerides, Lp(a) and remnant cholesterol, or an amount that reduces or eliminates a patient's need for lipoprotein apheresis or that reduces a patient's normalized rate of apheresis (as defined elsewhere herein).

In the case of an anti-PCSK9 antibody, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-PCSK9 antibody. According to certain exemplary embodiments of the present invention, a therapeutically effective amount of an anti-PCSK9 antibody is 75 mg, 150 mg or 300 mg (e.g., in the case of alirocumab), or 140 mg or 420 mg (e.g., in the case of evolocumab). Other dosing amounts of PCSK9 inhibitors will be apparent to persons of ordinary skill in the art and are contemplated within the scope of the present invention.

The amount of anti-PCSK9 antibody contained within the individual doses may be expressed in terms of milligrams of antibody per kilogram of patient body weight (i.e., mg/kg). For example, the anti-PCSK9 antibody may be administered to a patient at a dose of about 0.0001 to about 10 mg/kg of patient body weight.

### Combination Therapies

As described elsewhere herein, the methods of the present invention may comprise administering a PCSK9 inhibitor to a patient in combination with (“on top of”) the patient's previously prescribed lipid lowering therapy. For example, in the context of reducing or eliminating the need for lipoprotein apheresis, a PCSK9 inhibitor may be administered to a patient in combination with a stable daily therapeutic statin regimen. Exemplary daily therapeutic statin regimens that a PCSK9 inhibitor may be administered in combination with in the context of the present invention include, e.g., atorvastatin (10, 20, 40 or 80 mg daily), (atorvastatin/ezetimibe 10/10 or 40/10 mg daily), rosuvastatin (5, 10 or 20 mg daily), cerivastatin (0.4 or 0.8 mg daily), pitavastatin (1, 2 or 4 mg daily), fluvastatin (20, 40 or 80 mg daily), simvastatin (5, 10, 20, 40 or 80 mg daily), simvastatin/ezetimibe (10/10, 20/10, 40/10 or 80/10 mg daily), lovastatin (10, 20, 40 or 80 mg daily), pravastatin (10, 20, 40 or 80 mg daily), and combinations thereof. Other lipid lowering therapies that a PCSK9 inhibitor may be administered in combination with in the context of the present invention include, e.g., (1) an agent which inhibits cholesterol uptake and or bile acid re-absorption (e.g., ezetimibe); (2) an agent which increase lipoprotein catabolismosuch as niacin); and/or (3) activators of the LXR transcription factor that plays a role in cholesterol elimination such as 22-hydroxycholesterol.

According to certain embodiments of the present invention, methods are provided comprising administering a PCSK9 inhibitor (e.g., an anti-PCSK9 antibody such as alirocumab, evolocumab, bococizumab, lodelcizumab, or ralpancizumab) to a patient in combination with an inhibitor of angiopoietin-like protein 3 (e.g., an anti-ANGPTL3 antibody such as REGN1500), an inhibitor of angiopoietin-like protein 4 (e.g., an anti-ANGPTL4 antibody such as the anti-ANGPTL4 antibody referred to in U.S. Pat. No. 9,120,851 as “H1H268P” or “H4H284P”), or an inhibitor of angiopoietin-like protein 8 (e.g., an anti-ANGPTL8 antibody).

In the context of the methods of the present invention, additional therapeutically active component(s), e.g., any of the agents listed above or derivatives thereof, may be administered just prior to, concurrent with, or shortly after the administration of a PCSK9 inhibitor; (for purposes of the present disclosure, such administration regimens are considered the administration of a PCSK9 inhibitor “in combination with” an additional therapeutically active component). The present invention includes pharmaceutical compositions and methods of use thereof in which a PCSK9 inhibitor is co-formulated with one or more of the additional therapeutically active component(s) as described elsewhere herein.

### Administration Regimens

According to certain embodiments of the present invention, multiple doses of a PCSK9 inhibitor (i.e., a pharmaceutical composition comprising a PCSK9 inhibitor) may be administered to a subject over a defined time course (e.g., on top of a daily therapeutic statin regimen or other background lipid lowering therapy). The methods according to this aspect of the invention comprise sequentially administering to a subject multiple doses of a PCSK9 inhibitor. As used herein, “sequentially administering” means that each dose of PCSK9 inhibitor is administered to the subject at a different point in time, e.g., on different days separated by a predetermined interval (e.g., hours, days, weeks or months). The present invention includes methods which comprise sequentially administering to the patient a single initial dose of a PCSK9 inhibitor, followed by one or more secondary doses of the PCSK9 inhibitor, and optionally followed by one or more tertiary doses of the PCSK9 inhibitor.

The terms “initial dose,” “secondary doses,” and “tertiary doses,” refer to the temporal sequence of administration of the individual doses of a pharmaceutical composition comprising a PCSK9 inhibitor. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the PCSK9 inhibitor, but generally may differ from one another in terms of frequency of administration. In certain embodiments, however, the amount of PCSK9 inhibitor contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

According to exemplary embodiments of the present invention, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of antigen-binding molecule which is administered to a patient prior to the administration of the very next dose in the sequence with no intervening doses.

The methods according to this aspect of the invention may comprise administering to a patient any number of secondary and/or tertiary doses of a PCSK9 inhibitor. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2, 4, 6, 8 or more weeks after the immediately preceding dose. Similarly, in embodiments involving multiple tertiary doses, each tertiary dose may be administered at the same frequency as the other tertiary doses. For example, each tertiary dose may be administered to the patient 1 to 2, 4, 6, 8 or more weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

The present invention includes administration regimens comprising an up-titration option (also referred to herein as “dose modification”). As used herein, an “up-titration option” means that, after receiving a particular number of doses of a PCSK9 inhibitor, if a patient has not achieved a specified reduction in one or more defined therapeutic parameters, the dose of the PCSK9 inhibitor is thereafter increased. For example, in the case of a therapeutic regimen comprising administration of 75 mg doses of an anti-PCSK9 antibody to a patient at a frequency of once every two weeks, if after 8 weeks (i.e., 5 doses administered at Week 0, Week 2 and Week 4, Week 6 and Week 8), the patient has not achieved a serum LDL-C concentration of less than 70 mg/dL, then the dose of anti-PCSK9 antibody is increased to e.g., 150 mg administered once every two weeks thereafter (e.g., starting at Week 10 or Week 12, or later).

In certain embodiments, the anti-PCSK9 antibody is administered to a subject at a dose of about 75 mg every two weeks, for example for at least three doses (or throughout the course of the treatment regimen spanning days, weeks, months, or years).

In certain embodiments, the anti-PCSK9 antibody is administered to a subject at a dose of about 150 mg every two weeks, for example for at least three doses (or throughout the course of the treatment regimen spanning days, weeks, months, or years).

In some embodiments, the antibody is administered to a subject at a dose of about 75 mg every two weeks for 12 weeks, and the dose remains at 75 mg every two weeks if, at week 8, the subject's LDL-C value was less than 100 mg/dl and a 30% reduction of LDL-C.

In other embodiments, the antibody is administered to a subject at a dose of about 75 mg every two weeks for 12 weeks, and the dose is titrated up to about 150 mg every two weeks if, at week 8, the subject's LDL-C value was greater than or equal to 100 mg/dl.

In some embodiments, the antibody is administered to a subject at a dose of about 75 mg every two weeks for 12 weeks, and the dose remains at 75 mg every two weeks if, at week 8, the subject's LDL-C value was less than 70 mg/dl and a 30% reduction of LDL-C.

In another embodiment, the antibody is administered to a subject at a dose of about 300 mg every four weeks.

In a further embodiment, the antibody is administered to a subject at a dose of about 300 mg every four weeks for a total of three doses, and the dose is changed to 150 mg every two weeks for another 36 weeks if, at week 8, the subject did not achieve a pre-determined treatment goal or the subject did not have at least a 30% reduction of LDL-C from baseline.

In certain embodiments, the anti-PCSK9 antibody is administered to a subject at a dose of about 150 mg every four weeks for at least three doses.

In some embodiments, the antibody is administered to a subject at a dose of about 150 mg every four weeks for 12 weeks, and the dose remains at 150 mg every four weeks if, at week 8, the subject's LDL-C value was less than 100 mg/dl and a 30% reduction of LDL-C.

In other embodiments, the antibody is administered to a subject at a dose of about 150 mg every four weeks for 12 weeks, and the dose is titrated up to about 300 mg every two weeks if, at week 8, the subject's LDL-C value was greater than or equal to 100 mg/dl.

In some embodiments, the antibody is administered to a subject at a dose of about 150 mg every four weeks for 12 weeks, and the dose remains at 150 mg every four weeks for another 12 weeks if, at week 8, the subject's LDL-C value was less than 70 mg/dl and a 30% reduction of LDL-C.

In another embodiment, the antibody is administered to a subject at a dose of about 300 mg every four weeks.

In a further embodiment, the antibody is administered to a subject at a dose of about 300 mg every four weeks for a total of three doses, and the dose is changed to 150 mg every two weeks for another 36 weeks if, at week 8, the subject did not achieve a pre-determined treatment goal or the subject did not have at least a 30% reduction of LDL-C from baseline.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Generation of Human Antibodies to Human PCSK9

Human anti-PCSK9 antibodies were generated as described in U.S. Pat. No. 8,062,640. The exemplary PCSK9 inhibitor used in the following Example is the human anti-PCSK9 antibody designated “mAb316P,” also known as “REGN727,” or “alirocumab.” mAb316P has the following amino acid sequence characteristics: a heavy chain comprising SEQ ID NO:5 and a light chain comprising SEQ ID NO:9; a heavy chain variable region (HCVR) comprising SEQ ID NO:1 and a light chain variable domain (LCVR) comprising SEQ ID NO:6; a heavy chain complementarity determining region 1 (HCDR1) comprising SEQ ID NO:2, a HCDR2 comprising SEQ ID NO:3, a HCDR3 comprising SEQ ID NO:4, a light chain complementarity determining region 1 (LCDR1) comprising SEQ ID NO:7, a LCDR2 comprising SEQ ID NO:8 and a LCDR3 comprising SEQ ID NO:10.

### Example 2: A Randomized, Double-Blind, Placebo-Controlled, Parallel-Group Study to Evaluate the Efficacy and Safety of Alirocumab in Patients with Heterozygous Familial Hypercholesterolemia Undergoing Lipid Apheresis Therapy

### Introduction

The objective of the present study was to evaluate the effect of alirocumab 150 mg administered once every two weeks (“Q2W”) in comparison with placebo on the frequency of LDL apheresis treatments in patients with HeFH undergoing LDL apheresis therapy.

Adult men and women diagnosed with HeFH who were undergoing LDL apheresis therapy at a frequency of once every week or once every two weeks were enrolled in this study. The subjects' background treatment with LMT was maintained throughout the study. In such patients, the choice of placebo as a control is appropriate for the objectives of this study, since it provides the most robust assessment of efficacy and safety of alirocumab.

An average reduction of the LDL-C value ranges from 30% to 75% depending on the technique and duration of apheresis (Bambauer et al., Scientific World Journal, 2012, 2012:1-19). With weekly or bi-weekly treatment, the LDL-C levels can be reduced by 40% to 50%. According to the present study, apheresis was not performed when the LDL-C value at that visit was ≥30% lower than the baseline (pre-apheresis) LDL-C value. The LDL-C levels rise towards baseline after each apheresis procedure, but do not reach the original levels; with repeated weekly or bi-weekly apheresis, the baseline levels continue to fall until a plateau is reached (Thompsen et al., Atherosclerosis, 2006, 189:31-38). As such, a criterion to determine the necessity of apheresis, based on a 30% reduction in LDL-C levels, is justified since this reduction, if observed with administration of alirocumab, will be consistent over time, achieving approximately the same level of reduction in LDL-C as with the apheresis procedure.

Alirocumab Q2W (75 mg and 150 mg SC) and Q4W (150 mg and 300 mg SC) dosing regimens have been evaluated in other phase 3 trials of alirocumab. Based on the results of 2 dose finding studies, the Q2W dosing regimen was selected to maintain constant LDL-C lowering throughout the inter-dosing interval in all patients, with the maximum efficacy at 12 weeks provided by the 150 mg Q2W dosing. Heterozygous familial hypercholesterolemia patients generally have high baseline LDL-C levels. Considering that these patients have had life-long exposure to elevated LDL-C, their LDL-C target is either 100 mg/dL or 70 mg/dL, depending on their history of CVD or other risk factors. To reach and maintain their LDL-C target, it was surmised that such patients were very likely to require the most potent dose of alirocumab and at a frequency needed to maintain this effect at the end of the dosing interval. Therefore, the 150 mg Q2W dose was selected for this study.

In the double-blind treatment period of this study, 150 mg alirocumab Q2W administered for 18 weeks to patients with HeFH who require apheresis every 1 or 2 weeks to lower LDL-C, was evaluated for its ability to reduce the frequency of apheresis from week 7 to week 18 compared to the frequency during the 8 weeks before screening. Patients received 150 mg alirocumab Q2W through week 76 in the open-label treatment period.

Preliminary pharmacokinetic (PK) data from phase 2 studies, DFI11565, DFI11566, and R727-CL-1003, showed that exposure to alirocumab declined during the 8-week follow-up period that followed the double-blind treatment period, with total serum concentrations of alirocumab still detectable, but at very low levels. Therefore, to ensure sufficiently low, noneffective serum alirocumab concentrations, patients were followed during a follow-up period of 8 weeks (i.e., 10 weeks after the last dose).

### Study Objectives

The primary objective of the study was to evaluate the effect of alirocumab 150 mg Q2W in comparison with placebo on the frequency of LDL apheresis treatments in patients with HeFH undergoing weekly or bi-weekly LDL apheresis therapy.

The secondary objectives of the study were: (a) To evaluate the effect of alirocumab 150 mg Q2W on LDL-C levels in patients with HeFH undergoing LDL apheresis therapy; (b) To evaluate the effect of alirocumab 150 mg Q2W on the following lipid parameters: ApoB, non-HDL-C, total cholesterol, Lp(a), HDL-C, TGs, and ApoA-1 in patients with HeFH undergoing LDL apheresis therapy during the study; (c) To evaluate the safety and tolerability of alirocumab 150 mg Q2W in patients with HeFH undergoing LDL apheresis therapy; (d) To assess the PK of alirocumab 150 mg Q2W in patients with HeFH undergoing LDL apheresis therapy (QW versus Q2W); (e) To evaluate the development of anti-alirocumab antibodies; (f) To evaluate PCSK9 levels in response to alirocumab therapy as well as pre and post-apheresis; and (g) To evaluate quality of life (“QOL”) in these patients.

### Study Design

The present study was a randomized, double-blind, placebo-controlled, parallel-group study in patients with HeFH undergoing LDL apheresis therapy every 1 or 2 weeks.

The study consisted of 4 periods: screening, double-blind treatment period, open-label treatment period, and a follow-up. The double-blind treatment period comprised 2 intervals: day 1 to week 6 (with planned study drug administration on days 1, 15, and 29), when apheresis frequency will be fixed to the individual patient's established schedule, and week 7 to week 18 (with planned study drug administration on days 43, 57, 71, 85, 99, and 113), when apheresis frequency will be adjusted based on the patient's response to treatment.

Screening: Patients who had been on a stable apheresis schedule (every 7 or 14 days), with stable apheresis settings for at least 4 weeks prior to the screening visit (week −2) and stable background medical LMT for at least 8 weeks prior to the screening visit (week −2), entered a 2-week screening period.

Double-Blind Treatment: Patients who met all inclusion criteria and none of the exclusion criteria were randomly assigned in a 2:1 ratio to receive 18 weeks of treatment: Alirocumab 150 mg SC Q2W, or Placebo for alirocumab SC Q2W.

Treatment injections during the double-blind treatment period were administered Q2W starting on the day of randomization (week 0 [day 1]/visit 2). Patients were monitored at the clinical site for 30 minutes after the first dose. If LDL apheresis coincided with study drug administration, study drug was administered immediately after completion of the LDL apheresis procedure.

From day 1 to week 6, the apheresis frequency was fixed to the individual patient's established schedule (QW or Q2W). Starting at week 7, the LDL apheresis was administered based on the LDL-C value at that visit (determined by point-of-care testing). LDL apheresis was NOT administered when the LDL-C value at that visit was at least 30% lower than the pre-apheresis LDL-C value at baseline (day 1). The criterion to determine the necessity of apheresis, based on a 30% reduction in LDL-C levels, is justified since this reduction, if observed with administration of alirocumab, will be consistent over time, achieving approximately the same level of reduction in LDL-C as with the apheresis procedure. Investigators remained blinded to the point-of-care lab value for LDL-C and only received an alert as to whether or not LDL apheresis should be administered.

Open-label treatment: In the open-label treatment period, patients received alirocumab 150 mg SC Q2W. Treatment continued uninterrupted from the last dose of study drug during the double-blind treatment period (last dose at week 16) to week 18 (the first dose in the open-label treatment period), through week 76.

The first injection during the open-label treatment period was administered at the week 18 visit (the first visit of the open-label treatment period) as part of patient injection training. Subsequent injections were administered by the patient or a designated caregiver (spouse, relative, etc.) at a patient-preferred location (e.g., home or place of work). Patients were also permitted to choose to return to the site Q2W to have the injection administered by study personnel.

Apheresis treatment was not required in the open-label treatment period of the study. At the investigator's discretion, patients were permitted to continue to undergo apheresis procedures, as needed. In the event that a planned apheresis procedure coincided with a clinic visit or alirocumab dosing day, study assessments were done before apheresis, and alirocumab was administered after apheresis.

Follow-up: Patients were seen at the end of study visit at week 86.

Throughout the Study: All samples for clinical laboratory (lipid and specialty lipid panels) and PK evaluations were obtained immediately prior to and immediately after the LDL apheresis procedure (if LDL apheresis is administered at that visit) and before study drug administration.

For those patients not undergoing LDL apheresis at a visit, all samples for clinical laboratory evaluation were obtained before administration of study drug.

Overall safety was assessed by monitoring/evaluation of TEAEs, physical examinations, vital signs (pulse rate and blood pressure), electrocardiograms (ECGs), and clinical safety laboratory tests at prespecified time points. The potential emergence of anti-alirocumab antibodies was evaluated. PCSK9 levels were evaluated prior and post-apheresis.

The use of all medications and nutritional supplements (including consumption of red yeast products) known to alter serum lipids, including (but not limited to) statins, ezetimibe, fibrates, niacin, and bile acid resins was permitted, as long as that therapy had been stable for at least 8 weeks prior to the screening visit (week-2). Patients were instructed to continue taking their background medical LMT throughout the duration of the study, starting at screening through the end of treatment visit.

Patients' diets remained stable throughout the duration of the study, starting at screening through the end of treatment visit.

Patients' exercise regimens remained stable throughout the duration of the study, starting at screening through the end of treatment visit.

### Patient Selection

The study protocol called for approximately 63 patients to be enrolled at up to 15 sites in the US and Germany. The patients were randomized as follows: approximately one-third of enrolled patients (˜21) received placebo; and approximately two-thirds of enrolled patients (˜42) received alirocumab.

The study population consisted of adult men and women diagnosed with HeFH who were undergoing every 1 or 2 weeks LDL apheresis therapy.

Randomization was stratified according to: Apheresis frequency: QW vs Q2W; and baseline Lp(a) levels: normal (<30 mg/dL) vs elevated (≥30 mg/dL). Since some patients with elevated LDL-C also have elevated Lp(a) levels, stratification ensured adequate representation in the placebo/treatment groups.

Inclusion Criteria: The patients enrolled in this study were required to meet conditions 1-6 (below) to be eligible for inclusion in the study:
(1) Men and women ≥18 years of age at the time of the screening visit
(2) Diagnosis of HeFH; [Note: Diagnosis of HeFH was made either by genotyping or by clinical criteria. For those patients who had not been genotyped, the clinical diagnosis could be made based on either the Simon Broome criteria with a criteria for definite FH, or the WHO/Dutch Lipid Network criteria with a score >8 points];
(3) Currently undergoing LDL apheresis therapy QW for at least 4 weeks, or Q2W for at least 8 weeks prior to the screening visit (week −2) and have initiated apheresis treatment for at least 5 months prior to that; [Note: Acceptable apheresis techniques are: Double membrane filtration, Immunoadsorption, Heparin-induced LDL precipitation, Direct adsorption of lipids, Dextran sulfate adsorption (plasma), Dextran Sulfate adsorption (whole blood)];
(4) Willing and able to comply with clinic visits and study-related procedures;
(5) Provide signed informed consent; and
(6) Able to understand and complete study-related questionnaires.

Exclusion Criteria: Prospective patients who met any of the following criteria were excluded from the study:
(1) Homozygous FH;
(2) Background medical LMT (if applicable) that had not been stable for at least 8 weeks prior to the screening visit (week −2);
(3) LDL apheresis schedule/apheresis settings that had not been stable for at least 4 weeks prior to the screening visit (week −2) for patients undergoing apheresis weekly and at least 8 weeks prior to the screening visit (week −2) for patients undergoing apheresis bi-weekly;
(4) An LDL apheresis schedule other than QW to Q2W;
(5) Initiation of a new exercise program or exercise that has not remained stable within 8 weeks prior to the screening visit (week −2);
(6) Initiation of a new diet or a diet that has not been stable within 8 weeks prior to the screening visit (week −2);
(7) Use of nutraceuticals or over-the-counter therapies known to affect lipids, at a dose/amount that has not been stable for at least 8 weeks prior to the screening visit (week −2), or between the screening and randomization visit;
(8) Presence of any clinically significant uncontrolled endocrine disease known to influence serum lipids or lipoproteins;
(9) Signs and symptoms of hypothyroidism (thyroid replacement therapy is permitted);
(10) History of bariatric surgery within 12 months prior to the screening visit (week −2);
(11) Unstable weight (variation >5 kg) within 2 months prior to the screening visit (week −2);
(12) Newly diagnosed (within 3 months prior to randomization visit [day 1]) diabetes mellitus or poorly controlled (hemoglobin A1c [HbA1c]>9%) diabetes;
(13) Use of systemic corticosteroids, unless used as replacement therapy for pituitary/adrenal disease with a stable regimen for at least 6 weeks prior to randomization; topical, intra-articular, nasal, inhaled and ophthalmic steroid therapies are not considered as ‘systemic’ and are allowed;
(14) Use of estrogen or testosterone therapy, unless the regimen has been stable in the past 6 weeks prior to the screening visit (week −2) and no plans to change the regimen during the study;
(15) Systolic blood pressure >160 mm Hg or diastolic blood pressure >100 mm Hg at the screening visit (week −2) or time of randomization (day1) [Note: Blood pressure assessment for study eligibility may be obtained at a visit occurring between these 2 visits in the event that the patient had not taken, or planned not to take, prescribed hypertensive medications at the screening or randomization visit due to apheresis schedule];
(16) History of a myocardial infarction (MI), unstable angina leading to hospitalization, coronary artery bypass graft surgery (CABG), percutaneous coronary intervention (PCI), uncontrolled cardiac arrhythmia, carotid surgery or stenting, stroke, transient ischemic attack, or carotid revascularization within 3 months prior to the screening visit (week −2), or endovascular procedure or surgical intervention for peripheral vascular disease within 1 month prior to the screening visit (week −2);
(17) History of New York Heart Association Class III or IV heart failure within 12 months prior to the screening visit;
(18) Known history of a hemorrhagic stroke;
(19) History of cancer within the past 5 years, except for adequately treated basal cell skin cancer, squamous cell skin cancer, or in situ cervical cancer;
(20) Known history of a positive test for human immunodeficiency virus;
(21) Use of any active investigational drugs within 1 month or 5 half-lives of screening, whichever is longer;
(22) Patients who have been treated with at least 1 dose of alirocumab or any other anti-PCSK9 monoclonal antibody in any other clinical studies;
(23) Conditions/situations such as: (a) Any clinically significant abnormality identified at the time of screening that, in the judgment of the investigator or any sub-investigator, would preclude safe completion of the study or constrain endpoints assessment; eg, major systemic diseases, patients with short life expectancy; or (b) Considered by the investigator or any sub-investigator as inappropriate for this study for any reason, e.g.,: (i) Deemed unable to meet specific protocol requirements, such as scheduled visits; (ii) Deemed unable to tolerate injections, as per the patient or the investigator; (iii) Investigator or any sub-investigator, pharmacist, study coordinator, other study staff or relative thereof directly involved in the conduct of the protocol, etc; (iv) Presence of any other conditions (e.g., geographic or social), either actual or anticipated, that the investigator feels would restrict or limit the patient's participation for the duration of the study;
(24) Laboratory findings during screening period (not including randomization labs): (a) Positive test for hepatitis B surface antigen and/or hepatitis C antibody; (b) Positive serum beta-hCG or urine pregnancy test in women of childbearing potential; (c) TG>500 mg/dL (>5.65 mmol/L) (1 repeat lab is allowed); (d) eGFR<15 mL/min/1.73 m2 according to 4-variable Modification of Diet in Renal Disease Study equation (calculated by Central Lab); (e) Alanine aminotransferase (ALT) or aspartate aminotransferase (AST)>3× upper limit of normal (ULN) (1 repeat lab is allowed); (f) CPK>3×ULN (1 repeat lab is allowed);
(25) Known hypersensitivity to monoclonal antibody therapeutics or to any component of the drug product;
(26) Pregnant or breastfeeding women;
(27) Women of childbearing potential not protected by highly-effective method(s) of birth control (as defined in the informed consent form [ICF] and/or in a local protocol addendum) and/or who are unwilling or unable to be tested for pregnancy. Postmenopausal women must be amenorrheic for at least 12 months.

### Study Treatments

The study treatment was a single SC injection of 1 mL for a 150 mg dose of alirocumab or placebo, provided in an autoinjector or a prefilled syringe, administered in the abdomen, thigh, or outer area of the upper arm administered Q2W.

During the double-blind treatment period (day 1 to week 18), eligible patients were randomized to receive: Alirocumab 150 mg SC Q2W, or Placebo for alirocumab SC Q2W.

During the open-label treatment period patients received open-label alirocumab 150 mg SC Q2W, starting at week 18 with the last dose administered at week 76.

Sterile alirocumab drug product was supplied at a concentration of 150 mg/mL in histidine, pH 6.0, polysorbate 20, and sucrose in an autoinjector or a prefilled syringe.

Placebo matching alirocumab was supplied in the same formulation as alirocumab, without the addition of protein, in an autoinjector or a prefilled syringe.

All patients and/or caregivers who elected to inject the study drug outside of the clinic on dosing days were trained by the study staff before administering injections. Patients/caregivers were instructed on the administration of study drug at the week 18 visit, and self-administered the first dose of the open-label treatment period at the week 18 visit. Subsequent injections were administered Q2W by the patient (self-injection) or a designated caregiver (spouse, relative, etc.) at a patient-preferred location (e.g., home or place of work).

Patients were also given the option to return to the site Q2W to have the injection administered by study personnel.

### Method of Treatment Assignment

Patients were randomly assigned to receive placebo, or alirocumab 150 mg Q2W in a ratio of 1:2, implementing a permuted-block design to ensure even distribution of the treatment assignments. Randomization was stratified according to the frequency of the apheresis procedure (every 7 or 14 days) and Lp(a) levels (normal or elevated). Enrollment was capped so that no more than two-thirds of the patients were undergoing apheresis.

All patients, regardless of treatment assignment in the double-blind treatment period, received alirocumab 150 mg Q2W during the open-label treatment period.

### Concomitant Medications

Any treatment administered from the time of informed consent to the end of the follow-up period/final study visit is considered concomitant medication. This includes medications that were started before the study and were ongoing during the study. Concomitant medications were kept to a minimum during the study. If considered necessary for the patient's welfare and unlikely to interfere with study drug, concomitant medications (other than those that are prohibited during the study) were permitted to be given at the discretion of the investigator, with a stable dose (when possible).

Prohibited Medications: Any background medical LMT that was not stable for at least 8 weeks prior to the screening visit (week 2) was prohibited.

Permitted Medications: The use of all medications and nutritional supplements (including consumption of red yeast products) known to alter serum lipids including, but not limited to, statins, ezetimibe, fibrates, niacin, and bile acid resins was permitted as long as that therapy had been stable for at least 8 weeks prior to the screening visit (week 2). Patients were instructed to continue taking their background medical LMT throughout the duration of the study, starting at screening through the end of treatment visit.

Patients' diets remained stable throughout the duration of the study, starting at screening through the end of treatment visit.

Patients' exercise programs remained stable throughout the duration of the study, starting at screening through the end of treatment visit.

### Study Endpoints

Baseline characteristics will include standard demography (e.g., age, race, weight, height, etc.), disease characteristics including medical history, and medication history for each patient.

Primary Efficacy Endpoint: The primary efficacy endpoint is the rate of apheresis treatments during the 12-week period from week 7 to week 18, normalized by the number of planned apheresis treatments according to each patient's established schedule at screening, week −10 to week −2.

The normalized rate of apheresis is defined for each patient as: Number of actual apheresis treatments received from week 7 to week 18 divided by Number of planned apheresis treatments per randomization strata at baseline (6 for Q2W or 12 for QW).

In case of patient dropout before week 18, the actual number of apheresis procedures (from week 7 to week 18) will be added to the imputed number of the remaining planned apheresis treatments according to randomization strata for the numerator.

The impact of normalization on the planned apheresis treatments, according to each patient's established schedule at baseline, allows the homogenizing to the same rate scale for patients entering the study with different apheresis schedules (ie, apheresis QW or Q2W).

Secondary Efficacy Endpoints: For the following lipid endpoints, such as percent changes from baseline to a specific post-baseline visit during the double-blind treatment period, 2 time points are defined: prior to and after the apheresis procedure. For key efficacy endpoints, the lipid parameters will be analyzed using the assessments collected prior to each apheresis procedure. In the case where the apheresis is not performed at a protocol-specified visit, the single lab assessment planned for collection is used for both time points (i.e., including key efficacy endpoints). The baseline value is defined as the last available value for each of the parameters before the first double-blind dose of study drug.

Secondary endpoints of the present study were as follows:
(1) Percent change from baseline in LDL-C(pre-apheresis) to week 6, regardless of adherence to treatment;
(2) The standardized rate of apheresis treatments during the 4-week period from week 15 to week 18, defined similarly as for the primary efficacy endpoint;
(3) Percent change from baseline in ApoB (pre-apheresis) to week 6, regardless of adherence to treatment;
(4) Percent change from baseline in non-HDL-C(pre-apheresis) to week 6, regardless of adherence to treatment;
(5) Percent change from baseline in total cholesterol (pre-apheresis) to week 6, regardless of adherence to treatment;
(6) Percent change from baseline in ApoA-1 (pre-apheresis) to week 6, regardless of adherence to treatment;
(7) Proportion of patients with ≥30% reduction in LDL-C(pre-apheresis) at week 6, regardless of adherence to treatment;
(8) Proportion of patients with ≥50% reduction in LDL-C(pre-apheresis) at week 6, regardless of adherence to treatment;
(9) Percent change from baseline in LDL-C(pre-apheresis) to week 18, regardless of adherence to treatment;
(10) Percent change from baseline in ApoB (pre-apheresis) to week 18, regardless of adherence to treatment;
(11) Percent change from baseline in non-HDL-C(pre-apheresis) to week 18, regardless of adherence to treatment;
(12) Percent change from baseline in total cholesterol (pre-apheresis) to week 18, regardless of adherence to treatment;
(13) Percent change from baseline in ApoA-1 (pre-apheresis) to week 18, regardless of adherence to treatment;
(14) Proportion of patients with ≥30% reduction in LDL-C(pre-apheresis) at week 18, regardless of adherence to treatment;
(15) Proportion of patients with ≥50% reduction in LDL-C(pre-apheresis) at week 18, regardless of adherence to treatment;
(16) Change of W-BQ22 index score from baseline to week 18, regardless of adherence to treatment;
(17) Percent change from baseline in Lp(a) (pre-apheresis) to week 6, regardless of adherence to treatment;
(18) Percent change from baseline in HDL-C(pre-apheresis) to week 6, regardless of adherence to treatment;
(19) Percent change from baseline in TG levels (pre-apheresis) to week 6, regardless of adherence to treatment;
(20) Percent change from baseline in Lp(a) (pre-apheresis) to week 18, regardless of adherence to treatment;
(21) Percent change from baseline in HDL-C(pre-apheresis) to week 18, regardless of adherence to treatment;
(22) Percent change from baseline in TG levels (pre-apheresis) to week 18, regardless of adherence to treatment;
(23) The change in ApoB/ApoA-1 ratio (pre-apheresis) from baseline to week 6, regardless of adherence to treatment;
(24) The change in ApoB/ApoA-1 ratio (pre-apheresis) from baseline to week 18, regardless of adherence to treatment;
(25) The standardized rates of apheresis treatments during the 4-week period from week 7 to week 10, defined accordingly as for the primary efficacy endpoint;
(26) The standardized rates of apheresis treatments during the 4-week period from week 11 to week 14, defined accordingly as for the primary efficacy endpoint;
(27) Integrated assessment of frequency of apheresis and LDL-C values for 12-week period from the beginning of week 7 to the end of week 18 (patients ranked according to their LDL-C values and apheresis rate; the mean rank for all patients is determined and the difference of each treated patients LDL-C level or apheresis rate from mean rank for that variable is expressed as a percentage; the percent difference for the 2 variables on per patient basis are added to provide a summated percent difference. When combined and analyzed per treatment group, the resulting number will indicate low LDL-C and/or decreased rate of apheresis);
(28) Proportion of patients achieving LDL-C levels of <200 mg/dl, <130 mg/dL, <100 mg/dL and <70 mg/dL prior to apheresis at week 6, regardless of adherence to treatment;
(29) Proportion of patients achieving LDL-C levels of <200 mg/dL, <130 mg/dL, <100 mg/dL, and <70 mg/dL prior to apheresis at week 18, regardless of adherence to treatment;
(30) Raw values, as well as percent and absolute change in LDL-C and other lipids, from baseline to both time points of each visit, regardless of adherence to treatment, in the double-blind treatment period;
(31) Raw values, as well as percent and absolute change in LDL-C and other lipids, from baseline to each visit, regardless of adherence to treatment, in the open-label treatment period;
(32) Safety parameters (AEs [including adjudicated cardiovascular events], laboratory data, vital signs, and ECG) assessed throughout the study.

Other Endpoints: (1) Anti-alirocumab antibodies assessed throughout the study; (2) The percent change in high-sensitivity C-reactive protein (hs-CRP) and HbA1c from baseline to weeks 18 and 6; (3) Concentrations of total alirocumab in serum prior to and post-apheresis; (4) Free and total PCSK9 level prior and post-apheresis.

### Study Procedures

All laboratory samples were collected before the dose of study drug was administered. Blood samples were collected from patients in fasting conditions, if possible (e.g., overnight [at least 10 hours fast], only water) for all clinic visits for lipid panels. Although preferred, fasting was not a prerequisite for samples for the lipid panels. Alcohol consumption and smoking within 48 hours or intense physical exercise within 24 hours preceding blood sampling was not permitted.

Total-C, HDL-C, TG, ApoB, ApoA-1, and Lp(a) were directly measured by a central laboratory. LDL-C was calculated using the Friedewald formula. If TG values exceeded 400 mg/dl (4.52 mmol/L) then the central lab reflexively measured (via the beta quantification method) the LDL-C rather than calculating it. Non-HDL-C was calculated by subtracting HDL-C from the total-C. Ratio ApoB/ApoA-1 was calculated.

Lipid Panel (Fasting): Blood samples for the lipid panel (total-C, TG, HDL-C, and calculated LDL-C) were collected after at least a 10-hour fast at pre-specified time points.

Specialty Lipid Panel (Fasting): Blood samples for the specialty lipid panel (ApoB, ApoA-1, and Lp[a]) were collected after at least a 10-hour fast at pre-specified time points.

Well-Being Questionnaire: The W-BQ22 was used to assess the impact of hypercholesterolemia and treatment on well-being at specified time points.

Blood Pressure and Heart Rate: Blood pressure and heart rate were assessed at pre-specified time points. Blood pressure was preferably measured in sitting position under standardized conditions, approximately at the same time of the day, on the same arm, with the same apparatus (after the patient has rested comfortably in sitting position for at least 5 minutes). At the first screening visit, blood pressure was measured in both arms. The arm with the highest diastolic pressure was determined at this visit, and blood pressure was measured on this arm throughout the study. This highest value was recorded in the electronic case report form (eCRF). Heart rate was measured at the time of the measurement of blood pressure.

Physical Examination: A thorough and complete physical examination was performed at pre-specified time points.

Body Weight and Height: Body weight were obtained with the patient wearing undergarments or very light clothing and no shoes, and with an empty bladder. The same scale was preferably used throughout the study. The use of calibrated balance scales was recommended, if possible.

Electrocardiogram: Electrocardiograms were performed before blood is drawn during visits that required blood draws. A standard 12-lead ECG was performed at pre-specified time points. The 12-lead ECGs were performed after at least 10 minutes' rest and in the supine position. The electrodes were positioned at the same place insofar as possible, for each ECG recording throughout the study. The ECG was interpreted locally by the investigator. Any new and/or clinically significant changes in ECG parameters were immediately rechecked for confirmation before making any decision for the concerned patient. Any clinically significant abnormality was documented as an AE/SAE, as applicable. Each trace was analyzed in comparison with the screening recorded trace. All ECG traces were kept as source data. Heart rate was recorded from the ventricular rate, and the PR, QRS, RR and QT intervals will be recorded.

Laboratory Testing: All laboratory samples (including PK and ADA samples) were collected after assessments were performed and before a dose of study drug was administered at visits that correspond with a dosing day.

Adjudicating Cardiovascular Events: Adjudicated cardiovascular events included all cardiovascular AEs positively adjudicated. The adjudication categories were as follows: (1) CHD death; (2) Nonfatal MI; (3) Fatal and nonfatal ischemic stroke; (4) Unstable angina requiring hospitalization; (5) Congestive heart failure requiring hospitalization; (6) Ischemia-driven coronary revascularization procedure (PCI, CABG).

### Statistical Analyses

It was estimated that a sample size of 63 patients (alirocumab 42: placebo 21) would have at least 85% power to detect a 33% difference in mean apheresis rates using a two-sided significance level and assuming a standard deviation of 40%.

The primary efficacy analysis population was the primary intent-to-treat (ITT) population, defined as all randomized patients. The secondary efficacy analysis population was based on randomized patients who had least one pre-apheresis calculated LDL-C value before the first dose of study drug (or randomization, if the patient did not receive any study drug) and at least one calculated LDL-C value in one of the pre-apheresis analysis windows up to week 6. The statistical analysis was conducted when the last patient completed all efficacy assessments at week 18. LDL-C data were analysed post-hoc using the Kroon formula to estimate the interval means of LDL-C in weeks 6-18 (after potential withdrawal of apheresis therapy).

The standardized rate of apheresis treatments was analysed through the rate of treatments received during the 12-week period (week 7 to week 18), divided by the number of planned treatments (6 for Q2W and 12 for weekly), using the ranked analysis of covariance (ANCOVA) model. The standardized apheresis rate could range from 0 to 1, with 0 indicating that the patient skipped all planned apheresis treatments between week 7 to week 18, and 1 indicating that the patient received all planned treatments. A rate of 0.75 indicated that the patient received 75% of planned apheresis treatments (and skipped 25% of planned treatments). Patient drop-outs were accounted for according to pre-determined criteria. The median treatment difference was determined using the Hodges-Lehmann estimation and 95% confidence intervals (CI) using Moses distribution-free confidence interval.

A hierarchical inferential approach was used to control type I error. As the primary endpoint analysis was significant at the 5% alpha level, key secondary efficacy endpoints were tested sequentially.

### Results

### Baseline Characteristics

Baseline characteristics of the patients enrolled in the study are summarized in Tables 1 through 4.

TABLE 1
Patient Characteristics at Baseline

| | | | |
|---|---|---|---|
| | | Alirocumab | |
| | Placebo | 150 mg Q2W | All |
| Characteristic | (N = 21) | (N = 41) | (N = 62) |

Age

| | | | | | | |
|---|---|---|---|---|---|---|
| Mean (SD) | 57.0 | (10.5) | 59.5 | (9.2) | 58.7 | (9.7) |

| | | | |
|---|---|---|---|
| Min:Max | 27:73 | 41:79 | 27:79 |

Age group (years) [n (%)]

| | | | | | | |
|---|---|---|---|---|---|---|
| <65 | 16 | (76.2%) | 25 | (61.0%) | 41 | (66.1%) |
| >=65 | 5 | (23.8%) | 16 | (39.0%) | 21 | (33.9%) |

Sex [n (%)]

| | | | | | | |
|---|---|---|---|---|---|---|
| Male | 10 | (47.6%) | 26 | (63.4%) | 36 | (58.1%) |
| Female | 11 | (52.4%) | 15 | (36.6%) | 26 | (41.9%) |

Race [n (%)]

| | | | | | | |
|---|---|---|---|---|---|---|
| White | 21 | (100%) | 39 | (95.1%) | 60 | (96.8%) |

| | | | | | |
|---|---|---|---|---|---|
| Black or African | 0 | 2 | (4.9%) | 2 | (3.2%) |
| American | | | | | |

| | | | |
|---|---|---|---|
| Asian | 0 | 0 | 0 |
| Other, miscellaneous | 0 | 0 | 0 |

Country [n (%)]

| | | | | | | |
|---|---|---|---|---|---|---|
| Germany | 10 | (47.6%) | 20 | (48.8%) | 30 | (48.4%) |
| US | 11 | (52.4%) | 21 | (51.2%) | 32 | (51.6%) |

Weight (kg)

| | | | | | | |
|---|---|---|---|---|---|---|
| Mean (SD) | 86.9 | (21.3) | 90.0 | (16.1) | 88.9 | (17.9) |

| | | | |
|---|---|---|---|
| Min:Max | 56:124 | 60:124 | 56:124 |

BMI group (kg/m²) [n (%)]

| | | | | | | |
|---|---|---|---|---|---|---|
| >=30 | 8 | (38.1%) | 23 | (56.1%) | 31 | (50%) |

TABLE 2
Background LMT at Randomization

| | | | Alirocumab | | | |
|---|---|---|---|---|---|---|
| | Placebo | | 150 mg Q2W | | All | |
| Background LMT | (N = 21) | | (N = 41) | | (N = 62) | |
| Taking Statin | 13 | (61.9%) | 21 | (51.2%) | 34 | (54.8%) |
| Taking Maximum Daily | 8 | (38.1%) | 11 | (26.8%) | 19 | (30.6%) |
| Dose of Statin* | | | | | | |
| Any LMT other than | 16 | (76.2%) | 26 | (63.4%) | 42 | (67.7%) |
| Statins | | | | | | |

*Reasons for patients not being on statin or not on maximum daily dose of statin: muscle symptoms, increased CPK, elevated LFTs, concomitant medications, advance age, low BMI, glucose abnormalities, regional practice, potential for cognitive impairment.

TABLE 3
History of Apheresis Treatments

| | | |
|---|---|---|
| | Alirocumab | |
| Placebo | 150 mg Q2W | All |
| (N = 21) | (N = 41) | (N = 62) |

Frequency in which the subject is undergoing apheresis

| | | | | | | |
|---|---|---|---|---|---|---|
| QW | 9 | (42.9%) | 18 | (43.9%) | 27 | (43.5%) |
| Q2W | 12 | (57.1%) | 23 | (56.1%) | 35 | (56.5%) |

Time from first known apheresis treatment (years)

| | | | | | | |
|---|---|---|---|---|---|---|
| Mean (SD) | 8.32 | (8.11) | 7.22 | (7.60) | 7.59 | (7.73) |

| | | | |
|---|---|---|---|
| Median | 5.32 | 4.54 | 4.89 |
| Min:Max | 0.5:27.3 | 0.5:32.9 | 0.5:32.9 |

TABLE 4
Lipid Parameters at Baseline

| | | | |
|---|---|---|---|
| | | Alirocumab | |
| | Placebo | 150 mg Q2W | All |
| Lipid Parameter | (N = 21) | (N = 41) | (N = 62) |

Calculated LDL-C (mg/dL)

| | | | | | | |
|---|---|---|---|---|---|---|
| Mean (SD) | 191.6 | (68.9) | 175.1 | (54.6) | 180.7 | (59.7) |

| | | | |
|---|---|---|---|
| Median | 180.0 | 176.0 | 179.5 |
| Q1:Q3 | 140.0:240.0 | 129.0:219.0 | 133.0:227.0 |
| Min:Max | 81:316 | 53:275 | 53:316 |

Non-HDL-C (mg/dL)

| | | | | | | |
|---|---|---|---|---|---|---|
| Mean (SD) | 224.5 | (68.0) | 210.3 | (62.8) | 215.1 | (64.4) |

| | | | |
|---|---|---|---|
| Min:Max | 90:313 | 56:265 | 56:313 |

Total-C (mg/dL)

| | | | | | | |
|---|---|---|---|---|---|---|
| Mean (SD) | 272.3 | (73.6) | 256.9 | (60.6) | 262.1 | (65.1) |

| | | | |
|---|---|---|---|
| Min:Max | 147:399 | 140:377 | 140:399 |

HDL-C (mg/dL)

| | | | | | | |
|---|---|---|---|---|---|---|
| Mean (SD) | 47.8 | (17.3) | 46.6 | (16.2) | 47.0 | (16.4) |

| | | | |
|---|---|---|---|
| Min:Max | 24:98 | 24:108 | 24:108 |

TGs (mg/dL)

| | | | | | | |
|---|---|---|---|---|---|---|
| Mean (SD) | 164.4 | (61.6) | 176.0 | (87.2) | 172.1 | (79.0) |

| | | | |
|---|---|---|---|
| Min:Max | 81:308 | 59:393 | 59:393 |

Lipoprotein-(a) (mg/dL)

| | | | | | | |
|---|---|---|---|---|---|---|
| Mean (SD) | 45.7 | (49.5) | 43.0 | (54.5) | 43.9 | (52.4) |

| | | | |
|---|---|---|---|
| Min:Max | 7:202 | 1.5:285 | 21.5:285 |

Seventy-six patients with heFH undergoing regular weekly or Q2W lipoprotein apheresis provided consent to participate and were screened. As summarized in Tables 1-4, a total of 62 patients (age: mean±SD age, 58.7±9.7 years) were randomized. 58.1% of randomized patients were male. At screening visit, 28 patients were on no statin due to tolerability and 34 patients indicated taking daily statin with 19 of them taking the maximum daily dose. All patients had atherosclerotic disease (coronary and/or cerebrovascular and/or peripheral) and 56.5% had a family history of coronary heart disease. On average patients were on regular lipoprotein apheresis for (mean±SD) 7.6±7.7 years; median (Min:Max): 4.9 (0.5:32.9) years. The apheresis frequency was Q2W (56.5%) and QW (43.5%). 16.1% of patients had diabetes. 1.6% of patients had chronic kidney disease. Baseline mean LDL-C was 4.7 mmol/L (180.7 mg/dL); median (Min:Max): 4.7 (1.4:8.2) mmol/L [179.5 (53.0:316.0) mg/dL]. Baseline mean Lp(a) was 43.9 mg/dl (median [Min:Max]: 19.0 [1.5:285.0] mg/dL).

Sixty patients completed the 6-week double-blind treatment period (40 [97.6%] in the alirocumab group and 20 [95.2%] in the placebo group) (i.e. when apheresis rate was determined by the patient's established schedule), and 57 completed the 18-week double-blind treatment period (37 [90.2%] and 20 [95.2%], respectively), when the apheresis schedule was determined by LDL-C value previously achieved. Of the 5 (8.1%) patients who prematurely discontinued study treatment, 1 (4.8%) was on placebo (withdrawn due to adverse events) and 4 (9.8%) were on alirocumab (2 were withdrawn for adverse events and 1 for poor compliance, 1 patient withdrew consent).

The mean±standard deviation (SD) age of the population was 58.7±9.7 years, 36 (58.1%) patients were men, and 60 (96.8%) were white. The median (minimum, maximum) duration of apheresis treatments before entry into the study was 4.9 (0.5, 32.9) years.

The baseline characteristics were balanced between treatment groups. The mean calculated baseline LDL-C(at study entry) was 4.5±1.4 mmol/L (175.1 mg/dL) in the alirocumab group and 5.0±1.8 mmol/L (191.6 mg/dL) in the placebo group (P=0.35). Twenty-seven (43.5%) patients followed a weekly apheresis schedule at baseline and 35 (56.5%) patients followed a Q2W schedule. The mean LDL-C value in patients undergoing weekly apheresis was 3.9±1.3 mmol/L (151.3±51.3 mg/dL) versus 5.3±1.4 mmol/L (204.9±55.7 mg/dL) in patients undergoing Q2W apheresis. Thirty-eight (61.3%) patients had normal baseline lipoprotein(a) levels (<30 mg/dL) and 24 (38.7%) had elevated levels. Thirty-four (54.8%) patients were taking a statin at screening, 19 (55.9%) of whom were on a maximum daily dose. Baseline LDL-C values were (4.0±1.4 mmol/L (155.0±54.6 mg/dL) among statin-treated patients compared with 5.4±1.4 mmol/L (208.0±53.2 mg/dL) in patients not taking a statin. Thirty patients (48.4%) were from Germany and 32 (51.6%) from the United States.

The mean±SD duration of injection exposure was 17.4±2.3 weeks (8.6±1.3 injections) in the alirocumab group and 17.5±3.1 weeks (8.4±1.7 injections) in the placebo group.

### Efficacy Results

Efficacy results are summarized in Tables 5 through 8.

Table 5 shows the standardized rate of apheresis from week 7 to week 18

TABLE 5
Primary Efficacy Analysis: Standardized Rate of
Apheresis Treatments from Week 7 to Week 18 (ITT)

| | | Alirocumab |
|---|---|---|
| Standardized rate of apheresis | Placebo | 150 mg Q2W |
| treatments from week 7 to week 18 | (N = 21) | (N = 41) |
| Mean (SD) | 0.806 (0.191) | 0.128 (0.242) |
| Median | 0.833 | 0.000 |
| Min:Max | 0.42:1.00 | 0.00:1.00 |
| Hodges-Lehmann estimate of | | 0.750 |
| median treatment difference | | |
| 95% Moses distribution free CI | | (0.667 to 0.833) |
| p-value vs. placebo | | <.0001 |

Only legitimate apheresis treatment skipping per point-of-care LDL-C value is counted as “apheresis not occurred”. Missing apheresis treatment information (for any reason) from week 7 to week 18 is imputed to an outcome of the apheresis treatment “occurred” at the visit. The standardized rate of apheresis treatments is defined as: Number of actual apheresis treatments/planned apheresis treatments (6 for Q2W or 12 for QW).

TABLE 6
Standardized Rate of Apheresis Treatments
from Week 7 to Week 18 (ITT)

| | | Alirocumab | |
|---|---|---|---|
| Standardized rate of apheresis | Placebo | 150 mg Q2W | |
| treatments [n (%)] | (N = 21) | (N = 41) | |
| 0 | 0 | 26 | (63.4%) |
| >0 and <=0.25 | 0 | 7 | (17.1%) |
| >0.25 and <=0.5 | 3 (14.3%) | 5 | (12.2%) |
| >0.5 and <=0.75 | 5 (23.8%) | 1 | (2.4%) |
| >0.75 and <1 | 7 (33.3%) | 1 | (2.4%) |
| 1 | 6 (28.6%) | 1 | (2.4%) |

Only legitimate apheresis treatment skipping per point-of-care LDL-C value is counted as “apheresis not occurred”. Missing apheresis treatment information (for any reason) from week 7 to week 18 is imputed an outcome of the apheresis treatment “occurred” at the visit. Two patients (1 in placebo and 1 in alirocumab) terminated study treatments before week 6 and the standardized apheresis rates were imputed as 1.

The primary efficacy endpoint achieved statistically significant benefit in favour of alirocumab-treated patients, with a Hodges-Lehmann median estimate of the treatment difference versus placebo of 0.75 (95% confidence interval: 0.67-0.83). Therefore, alirocumab-treated patients had a 0.75 (75%) additional reduction in the standardized rate of apheresis treatments versus placebo-treated patients (P<0.0001). The median treatment difference in the standardized rate of apheresis treatments from week 7 to week 18, when apheresis treatment was determined by LDL-C concentration previously achieved, was 0.75 (95% CI 0.58-0.92) for patients who had been undergoing weekly apheresis and 0.67 (95% CI 0.50-1.00) for those on a Q2W schedule, in favour of alirocumab.

The median treatment difference in the standardized rate of apheresis treatments over the 4-week period (week 15 to week 18) was 0.50 (95% CI 0.50 to 1.00; P<0.0001) in favour of alirocumab, indicating a 50% reduction in the standardized rate of apheresis treatments versus placebo.

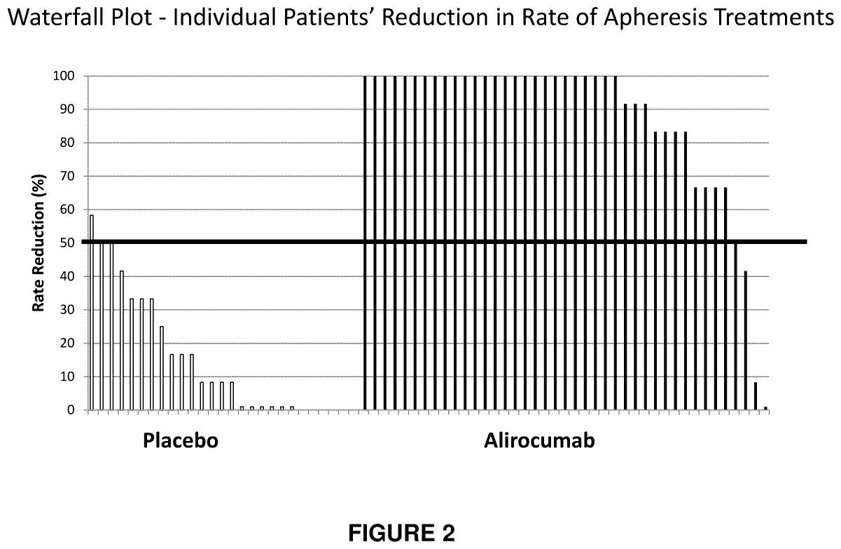

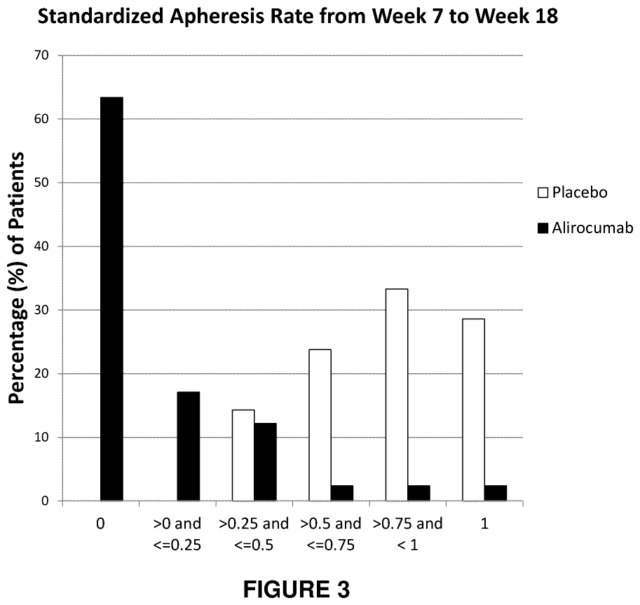

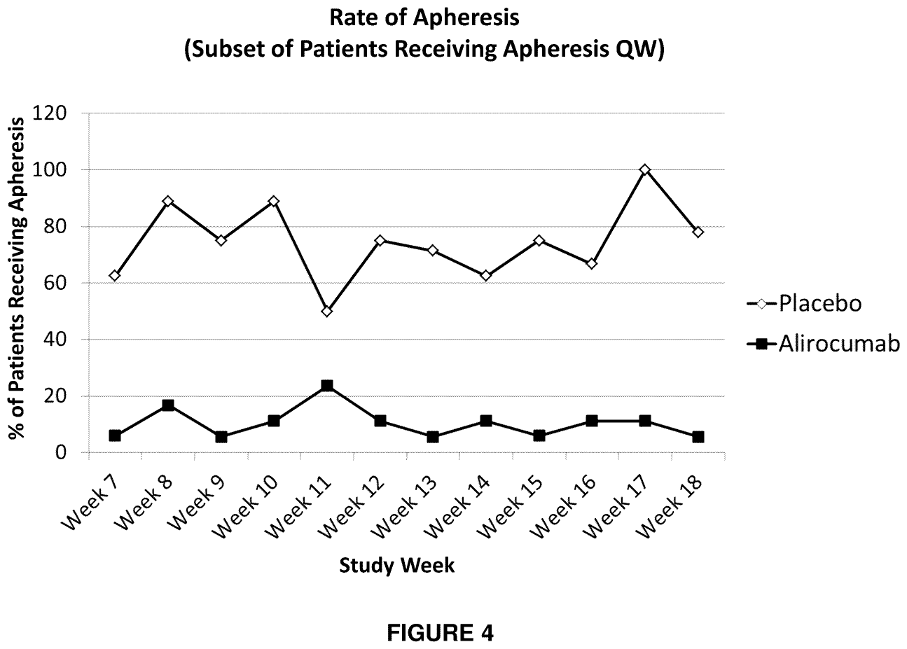

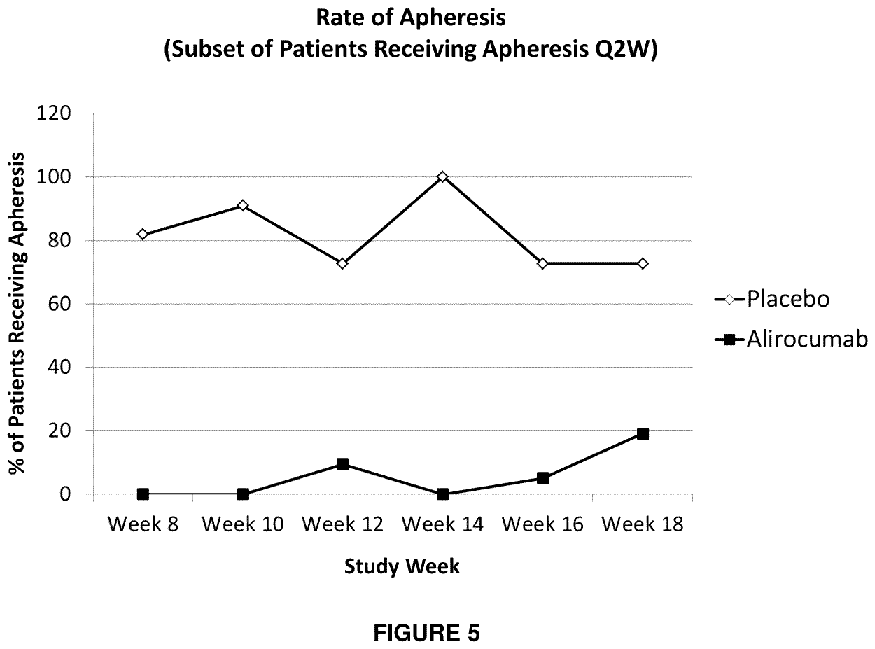

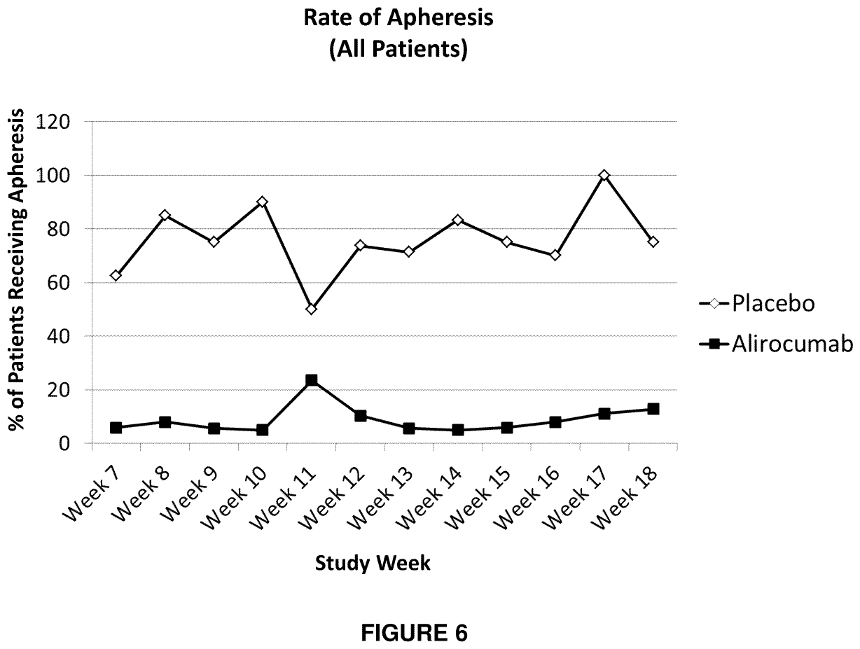

As shown in Table 5, the mean rate of apheresis treatments in alirocumab-treated patients was 0.128, whereas the mean rate of apheresis treatments in the placebo group was 0.806. A graphical representation of the percent reduction in rate of apheresis treatments for individual alirocumab-treated patients compared with individual patients in the placebo group over the 12-week period is shown in FIG. 2. During this period, 63.4% of patients on alirocumab had no apheresis procedures, and 92.7% avoided at least half of the procedures. That is, 26 patients in the alirocumab-treated group achieved a 100% reduction in rate of apheresis treatments. By contrast, none of the patients in the placebo group achieved a 100% reduction in rate of apheresis treatments. These results are also reflected in Table 6 and FIGS. 3 through 6, illustrating the rate of apheresis treatments for patients in placebo and alirocumab-treated groups from week 7 to week 18 of the study. The results show that patients who added alirocumab to their existing treatment regimen were able to significantly reduce the frequency of their standardized apheresis therapy over 12 weeks by 75% compared to placebo (P<0.0001).

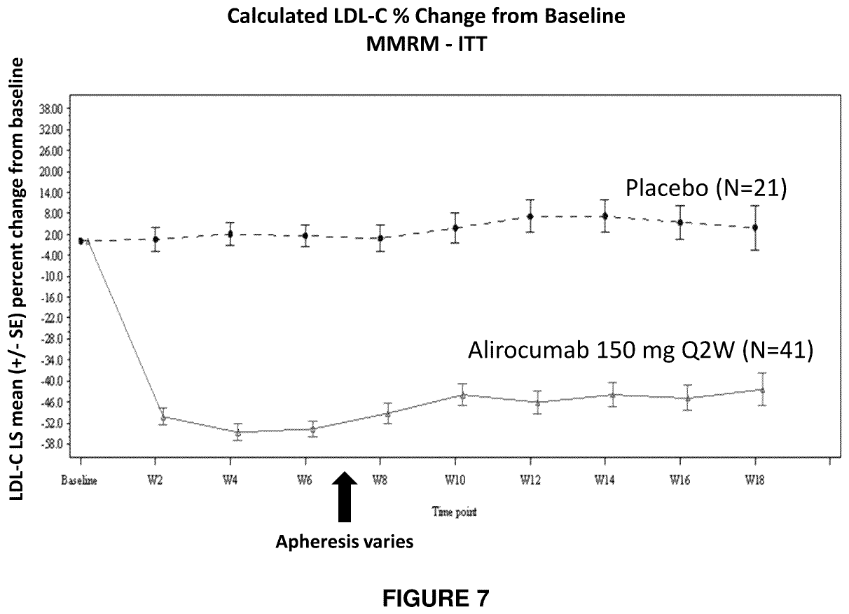

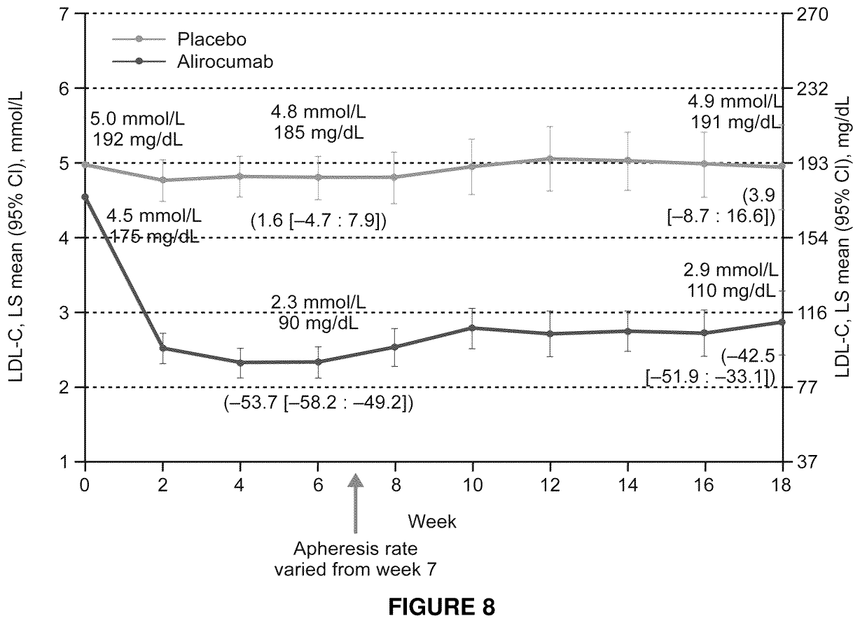

Table 7 and FIGS. 7 and 8 show the effects of alirocumab treatment as compared to placebo in terms of changes in serum LDL-C levels over the course of the study. Overall, alirocumab-treated patients exhibited a mean reduction in LDL-C from baseline greater than 50% at week 6, while patients in the placebo group did not exhibit any significant degree of LDL-C reductions.

TABLE 7
LDL-C Percent Change from Baseline Analysis
at Week 6 (pre-apheresis): MMRM - ITT

| | | |
|---|---|---|
| | | Alirocumab |
| Percent change from baseline in | Placebo | 150 mg Q2W |
| Calculated LDL Cholesterol | (N = 21) | (N = 41) |

Baseline (mg/dL)

| | | | | |
|---|---|---|---|---|
| Mean (SD) | 191.6 | (68.9) | 175.1 | (54.6) |

| | | |
|---|---|---|
| Median | 180.0 | 176.0 |
| Min:Max | 81:316 | 53:275 |

Week 6 percent change from baseline (%)

| | | | | |
|---|---|---|---|---|
| LS Mean (SE) | 1.6 | (3.1) | −53.7 | (2.3) |
| LS mean difference (SE) vs placebo | | | −55.3 | (3.9) |

| | |
|---|---|
| 95% CI | (−63.1 to −47.5) |
| p-value vs placebo | <.0001 |

Note:
Least-squares (LS) means, standard errors (SE) and p-value taken from MMRM (mixed-effect model with repeated measures) analysis. The model includes the fixed categorical effects of treatment group, randomization strata as per IVRS, time point, treatment-by-time point and strata-by-time point interaction, as well as the continuous fixed covariates of baseline calculated LDL-C value and baseline calculated LDL-C value-by-time point interaction. Time points in the model include bi-weekly timepoints from week 2 (pre-apheresis) to week 18 (pre-apheresis).

Key secondary efficacy results for placebo and alirocumab-treated patients are summarized in Table 8.

TABLE 8
Key Secondary Efficacy Analysis Results

| | Placebo | Alirocumab | | |
|---|---|---|---|---|
| Endpoints | Result | Result | Comparison | P-value |
| Calculated LDL-C | LS mean: 1.6% | LS mean: −53.7% | Diff: −55.3% | <0.0001 |
| Percent change from baseline to | | | | |
| Week 6 (Pre-apheresis) | | | | |
| Rate of apheresis treatments | Median: 1.000 | Median: 0.000 | Diff*: 0.500 | <0.0001 |
| Week 15 to week 18 | | | | |
| Apo B | LS mean: 1.2% | LS mean: −42.8% | Diff: −44.0% | <0.0001 |
| Percent change from baseline to | | | | |
| Week 6 (Pre-apheresis) | | | | |
| non-HDL-C | LS mean: 2.8% | LS mean: −47.1% | Diff: −50.0% | <0.0001 |
| Percent change from baseline to | | | | |
| Week 6 (Pre-apheresis) | | | | |
| Total-C | LS mean: 3.1% | LS mean: −36.4% | Diff: −39.4% | <0.0001 |
| Percent change from baseline to | | | | |
| Week 6 (Pre-apheresis) | | | | |
| Apo A-1 | LS mean: 0.0% | LS mean: 4.2% | Diff: 4.2% | 0.3012 |
| Percent change from baseline to | | | | |
| Week 6 (Pre-apheresis) | | | | |
| ≥30% reduction in LDL-C | Proportion: 4.8% | LS mean: 95.1% | Odds Ratio: 144.4 | <0.0001 |
| from baseline to | | | | |
| Week 6 (Pre-apheresis) | | | | |
| ≥50% reduction in LDL-C | Proportion: 0 | LS mean: 63.4% | Odds Ratio: 48.1 | 0.0006 |
| from baseline to | | | | |
| Week 6 (Pre-apheresis) | | | | |
| Calculated LDL-C | LS mean: 3.9% | LS mean: −42.5% | Diff: −46.4% | <0.0001 |
| Percent change from baseline to | | | | |
| Week 18 (Pre-apheresis) | | | | |
| Apo B | LS mean: 1.8% | LS mean: −33.5% | Diff: −35.2% | <0.0001 |
| Percent change from baseline to | | | | |
| Week 18 (Pre-apheresis) | | | | |
| non-HDL-C | LS mean: 4.7% | LS mean: −45.8% | Diff: −40.4% | <0.0001 |
| Percent change from baseline to | | | | |
| Week 18 (Pre-apheresis) | | | | |
| Total-C | LS mean: 4.6% | LS mean: −27.3% | Diff: −31.9% | <0.0001 |
| Percent change from baseline to | | | | |
| Week 18 (Pre-apheresis) | | | | |
| Apo A-1 | LS mean: −0.7% | LS mean: 8.1% | Diff: 8.8% | 0.0404 |
| Percent change from baseline to | | | | |
| Week 18 (Pre-apheresis) | | | | |
| ≥30% reduction in LDL-C | Proportion: 0 | LS mean: 65.9% | Odds Ratio: 39.8 | <0.0001 |
| from baseline to | | | | |
| Week 18 (Pre-apheresis) | | | | |
| ≥50% reduction in LDL-C | Proportion: 0 | LS mean: 43.9% | Odds Ratio: 18.8 | <0.0001 |
| from baseline to | | | | |
| Week 18 (Pre-apheresis) | | | | |
| W-BQ22 (Well-being measure) | LS mean: −0.34 | LS mean: −0.86 | Diff: −0.52 | 0.6887 |
| Change from baseline to | | | | |
| Week 18 | | | | |
| Lp(a) | LS mean: −4.0% | LS mean: −18.0% | Diff: −14.1% | 0.0262 |
| Percent change from baseline to | | | | |
| Week 6 (Pre-apheresis) | | | | |
| HDL-C | LS mean: 4.0% | LS mean: 9.2% | Diff: 5.2% | 0.2168 |
| Percent change from baseline to | | | | |
| Week 6 (Pre-apheresis) | | | | |
| TG | LS mean: 3.0% | LS mean: −12.9% | Diff: −15.8% | 0.0204 |
| Percent change from baseline to | | | | |
| Week 6 (Pre-apheresis) | | | | |
| Lp(a) | LS mean: −0.9% | LS mean: −5.0% | Diff: −4.1% | 0.6805 |
| Percent change from baseline to | | | | |
| Week 18 (Pre-apheresis) | | | | |
| HDL-C | LS mean: 2.5% | LS mean: 10.5% | Diff: 8.0% | 0.2022 |
| Percent change from baseline to | | | | |
| Week 18 (Pre-apheresis) | | | | |
| TG | LS mean: 4.2% | LS mean: −1.4% | Diff: −5.6% | 0.5770 |
| Percent change from baseline to | | | | |
| Week 18 (Pre-apheresis) | | | | |

The mean pre-apheresis LDL-C value decreased from 4.5 mmol/L (175 mg/dL) at baseline to 2.3 mmol/L (90 mg/dL) at week 6 in the alirocumab group; corresponding data for patients in the placebo group are 5.0 mmol/L (192 mg/dL) and 4.8 mmol/L (185 mg/dL) (FIG. 2). The LS mean±SE (95% CI) percent change in pre-apheresis LDL-C from baseline at week 6 was −53.7±2.3 (−58.2 to −49.2) in the alirocumab group and 1.6±3.1 (−4.7 to 7.9) in the placebo group (LS mean±SE percent difference −55.3±3.9, 95% CI −63.1 to −47.5; P<0.0001). By week 18, the mean LDL-C value in the alirocumab group had risen slightly, to 2.9 mmol/L (110 mg/dL), compared with 4.9 mmol/L (191 mg/dL) in the placebo group (LS mean±SE percent difference −46.4±7.9, 95% CI −62.3 to −30.5; P<0.0001).

A cross-validation, comparing point-of-care LDL-C values with central laboratory values, showed that both measures were highly correlated (Pearson's correlation 0.86).

Percent change from baseline to week 18 in lipoprotein(a) was −5.7% for alirocumab versus −3.0% for placebo among patients with normal baseline values, and 4.9% versus 7.6%, respectively, in patients with elevated baseline values. During weeks 7-18, when apheresis treatment could be withdrawn, alirocumab treatment was associated with a lower mean±SD time-averaged LDL-C value (using the Kroon formula 18) over the course of the (potential) apheresis interval of 2.4±1.3 mmol/L (92.7±50.2 mg/dL) versus 3.8±1.7 mmol/L (146.7±65.6 mg/dL) for placebo (P<0.0001).

In a post-hoc analysis, the time-averaged LDL-C values in the alirocumab-treated patients were consistently lower than those in the placebo-treated patients.

### Safety

TEAEs were reported by 75.6% of patients in the alirocumab group and by 76.2% of patients in the placebo group, none of which were fatal. The rates of serious adverse events (9.8% for alirocumab and 9.5% for placebo) and events leading to treatment discontinuation (4.9% and 4.8%, respectively) were also similar in both groups.

Three patients (7.3%) in the alirocumab group and none in the placebo group had two consecutive pre-apheresis calculated LDL-C values <0.7 mmol/L (25 mg/dL). Two of these patients had at least one adverse event, one of which had several serious adverse events (pneumonia, acute myocardial infarction, acute respiratory failure, cardiac failure congestive, sepsis and aortic valve stenosis). Twenty-seven patients (23 [56.1%] on alirocumab and 4 [19.0%] on placebo) had two consecutive LDL-C values <0.7 mmol/L after apheresis, of which 15 (65.2%) and 2 (50.0%), respectively, had an adverse event. None of the events was serious but one patient (4.3%) discontinued treatment with alirocumab.

### SUMMARY AND CONCLUSIONS

The present study shows that patients receiving alirocumab exhibited a reduced rate of apheresis treatments, and a reduced number of apheresis treatments over the course of the study, compared to patients receiving placebo. In particular, alirocumab treatment resulted in a higher reduction in the rate of apheresis treatments as compared to the placebo treatment, by a median estimate of 0.75 or 75% (p<0.0001).

The present study also demonstrated that 63.4% of patients treated with alirocumab had no apheresis performed vs. 0% in placebo treated patients (that is, no patients in the placebo-treated group were able to forego apheresis treatments completely, whereas 63.4% of patients in the alirocumab treatment arm were able to eliminate apheresis treatments altogether following alirocumab treatment). In addition, 92.7% of patients treated with alirocumab had at least 50% reduction in frequency of apheresis vs 14.3% in placebo treated patients. Moreover, alirocumab reduced LDL-C by 55% (vs. PBO) at week 6 from 175 mg/dl to 89.5 mg/dl. Significant reductions in apoB, non-HDL-C and TC were also observed in alirocumab-treated patients. Alirocumab treatment reduced the standardized rate of apheresis treatments from week 15 to week 18.

With regard to safety, subcutaneous administration of alirocumab in patients with HeFH undergoing LDL apheresis therapy every 1 or 2 weeks was generally safe and well tolerated. The number of patients reporting TEAEs overall and AESIs were comparable across treatment groups.

All of the patients in this study were at high cardiovascular risk and had taken LLT previously, including statins. At screening, only 54.8% of the patients were taking a statin, 55.9% of whom were on a maximally tolerated dose. A large proportion of the overall population reported a history of down-titration of statin treatment due to tolerability issues (43.5%) and 62.9% reported changing to a different statin. Various reasons were given for not taking a statin or for not taking it at the maximum daily dose, ranging from muscle symptoms to anxiety about side-effects, and regional practices/local labelling, indicating that patients with heFH on apheresis present a diverse and difficult-to-treat population, with limited treatment options.

Patients in the USA showed different characteristics to those in Germany, with higher baseline LDL-C, greater prevalence of statin intolerance, and a less frequent apheresis regimen. In the USA, lipoprotein apheresis for heFH is often only considered for patients who, after 6 months, do not have an adequate response to maximum tolerated drug therapy and have elevated LDL-C plus other cardiovascular risk factors. In Germany, where apheresis centers are more common, apheresis is considered within 12 months of failure of diet and LLT, and at a lower LDL-C threshold, and weekly is preferred to Q2W apheresis. Furthermore, recommendations for LDL-C lowering from Europe are based on a risk-stratified treat-to-target approach, whereas US guidelines advocate a dose-adapted approach. In view of the approach adopted in European guidelines, in which the target LDL-C value is <1.8 mmol/L (70 mg/dL), German patients on alirocumab with LDL-C above this target value would still meet the criteria for apheresis. Consequently, LLT with alirocumab may prove complementary to lipoprotein apheresis in patients with very high LDL-C or who fail to meet the target LDL-C value.

In conclusion, this study achieved significant reduction in the primary efficacy endpoint and demonstrates that PCSK9 inhibitors such as alirocumab are an effective therapeutic option to reduce or eliminate a patient's need for lipoprotein apheresis therapy, or to delay the requirement for such treatments.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. A method for eliminating a patient's need for lipoprotein apheresis therapy, or reducing the frequency of lipoprotein apheresis therapy required by a patient to achieve a target lipoprotein level, the method comprising selecting a patient with hypercholesterolemia who is being or has been treated with lipoprotein apheresis at an initial (pre-treatment) frequency, and administering one or more doses of an antibody or antigen-binding fragment thereof that specifically binds PCSK9 and that comprises a heavy chain variable region (HCVR) that comprises the heavy chain complementarity determining regions (CDRs) of a HCVR comprising the amino acid sequence set forth in SEQ ID NO: 1; and a light chain variable region (LCVR) that comprises the light chain CDRs of a LCVR comprising the amino acid sequence set forth in SEQ ID NO: 6, and one or more selected from the group consisting of: atorvastatin, atorvastatin & ezetimibe, rosuvastatin, cerivastatin, pitavastatin, fluvastatin, lovastatin, simvastatin, simvastatin & ezetimibe, pravastatin, and combinations thereof, to the patient, thereby lowering the level of at least one lipoprotein in the serum of the patient and reducing frequency of lipoprotein apheresis required by the patient to achieve a target lipoprotein level.

2. The method of claim 1, wherein the initial (pre-treatment) frequency of apheresis is once a week or once every two weeks.

3. The method of claim 1, wherein the frequency of apheresis following administration of the one or more doses of the antibody or antigen-binding fragment thereof that specifically binds PCSK9 is once every three weeks, once every four weeks, once every five weeks, or less frequent than once every five weeks.

4. The method of claim 1, wherein, following administration of the one or more doses of the antibody or antigen-binding fragment thereof that specifically binds PCSK9, the patient no longer requires apheresis to maintain the target lipoprotein level.

5. The method of claim 1, wherein the patient, prior to treatment with the one or more doses of the antibody or antigen-binding fragment thereof that specifically binds PCSK9, is diagnosed with heterozygous familial hypercholesterolemia (HeFH) or homozygous familial hypercholesterolemia (HoFH), and/or elevated lipoprotein(a) (Lp[a]).

6. The method of claim 1, wherein the lipoprotein apheresis therapy is selected from the group consisting of: cascade filtration, immunoadsorption, heparin-induced LDL precipitation, LDL-adsorption (dextran sulfate) liposorber, LDL hemoperfusion, and LDL-hemoperfusion (liposorber D).

7. The method of claim 1, wherein the patient, prior to or at the time of treatment with the one or more doses of the antibody or antigen-binding fragment thereof that specifically binds PCSK9, is on a stable lipoprotein apheresis schedule at the initial (pre-treatment) frequency for at least 2 weeks prior to administration of the first dose of the antibody or antigen-binding fragment thereof that specifically binds PCSK9.

8. The method of claim 1, wherein the patient is on a stable background lipid modifying therapy (LMT) prior to administration of the one or more doses of the antibody or antigen-binding fragment thereof that specifically binds PCSK9.

9. The method of claim 1, wherein the patient is on a stable background lipid modifying therapy (LMT) concurrent with administration of the one or more doses of the antibody or antigen-binding fragment thereof that specifically binds PCSK9.

10. The method of claim 8, wherein the stable background LMT is low-, moderate-, or high-dose statin therapy.

11. The method of claim 9, wherein the stable background LMT is low-, moderate-, or high-dose statin therapy.

12. The method of claim 1, wherein the lipoprotein that is lowered in the serum of the patient following administration of the one or more doses of the antibody or antigen-binding fragment thereof that specifically binds PCSK9 is one or more lipoproteins selected from the group consisting of LDL-C, ApoB, non-HDL-C, total cholesterol, and Lp(a).

13. The method of claim 1, wherein the target lipoprotein level is a serum LDL-C level of less than 200 mg/dL, less than 130 mg/dL, less than 100 mg/dL, or less than 70 mg/dL.

14. (canceled)

15. The method of claim 1, wherein the antibody or antigen-binding protein that specifically binds PCSK9 is administered to the patient at a dose of about 75 mg at a frequency of once every two weeks.

16. The method of claim 1, wherein the antibody or antigen-binding protein that specifically binds PCSK9 is administered to the patient at a dose of about 150 mg at a frequency of once every two weeks.

17. The method of claim 1, wherein the antibody or antigen-binding protein that specifically binds PCSK9 is administered to the patient at a dose of about 300 mg at a frequency of once every four weeks.

18-20. (canceled)

21. The method of claim 1, wherein the antibody or antigen-binding protein is alirocumab.

22. (canceled)

23. The method of claim 1, wherein the antibody or antigen binding fragment thereof comprises the heavy and light chain CDRs of a HCVR/LCVR amino acid sequence pair comprising SEQ ID NOs: 1/6.

24. The method of claim 23, wherein the antibody or antigen-binding fragment thereof comprises heavy and light chain CDR amino acid sequences having SEQ ID NOs:2, 3, 4, 7, 8 and 10.

25. The method of claim 24, wherein the antibody or antigen-binding fragment thereof comprises an HCVR having the amino acid sequence of SEQ ID NO:1 and an LCVR having the amino acid sequence of SEQ ID NO:6.


SEQUENCE LISTING (198 sequences)

SEQ ID NO: 1 - protein, 118 aa, synthetic construct [sequence omitted]

SEQ ID NO: 2 - protein, 8 aa, synthetic construct
  REGION 1..8: note = Description of Artificial Sequence: Synthetic peptide
GFTFNNYA

SEQ ID NO: 3 - protein, 8 aa, synthetic construct
  REGION 1..8: note = Description of Artificial Sequence: Synthetic peptide
ISGSGGTT

SEQ ID NO: 4 - protein, 11 aa, synthetic construct
  REGION 1..11: note = Description of Artificial Sequence: Synthetic peptide
AKDSNWGNFDL

SEQ ID NO: 5 - protein, 447 aa, synthetic construct
  REGION 1..447: note = Description of Artificial Sequence: Synthetic REGN727 heavy chain polypeptide
EVQLVESGGGLVQPGGSLRLSCAASGFTFNNYAMNWVRQAPGKGLDWVSTISGSGGTTNYADSVKGRFIISRDSSKHTLYLQMNSLRAEDTAVYYCAKDSNWGNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 113 aa, synthetic construct [sequence omitted]

SEQ ID NO: 7 - protein, 12 aa, synthetic construct
  REGION 1..12: note = Description of Artificial Sequence: Synthetic peptide
QSVLYRSNNRNF

SEQ ID NO: 8 - skipped

SEQ ID NO: 9 - protein, 220 aa, synthetic construct
  REGION 1..220: note = Description of Artificial Sequence: Synthetic REGN727 light chain polypeptide
DIVMTQSPDSLAVSLGERATINCKSSQSVLYRSNNRNFLGWYQQKPGQPPNLLIYWASTRESGVPDRFSGSGSGTDFTLTISSLQAEDVAVYYCQQYYTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 9 aa, synthetic construct
  REGION 1..9: note = Description of Artificial Sequence: Synthetic peptide
QQYYTTPYT

SEQ ID NO: 11 - protein, 127 aa, synthetic construct
  REGION 1..127: note = Description of Artificial Sequence: Synthetic polypeptide
EMQLVESGGGLVQPGGSLRLSCAASGFTFSSHWMKWVRQAPGKGLEWVANINQDGSEKYYVDSVKGRFTISRDNAKNSLFLQMNSLRAEDTAVYYCARDIVLMVYDMDYYYYGMDVWGQGTTVTVSS

SEQ ID NO: 12 - protein, 8 aa, synthetic construct
  REGION 1..8: note = Description of Artificial Sequence: Synthetic peptide
GFTFSSHW

SEQ ID NO: 13 - protein, 8 aa, synthetic construct
  REGION 1..8: note = Description of Artificial Sequence: Synthetic peptide
INQDGSEK

SEQ ID NO: 14 - protein, 20 aa, synthetic construct
  REGION 1..20: note = Description of Artificial Sequence: Synthetic peptide
ARDIVLMVYDMDYYYYGMDV

SEQ ID NO: 15 - protein, 112 aa, synthetic construct
  REGION 1..112: note = Description of Artificial Sequence: Synthetic polypeptide
DIVMTQSPLSLPVTPGEPASISCRSSQSLLHSNGNNYLDWYLQKPGQSPQLLIYLGSNRASGVPDRFSGSGSGTDFTLKISRVEAEDVGVYYCMQTLQTPLTFGGGTKVEIK

SEQ ID NO: 16 - protein, 11 aa, synthetic construct
  REGION 1..11: note = Description of Artificial Sequence: Synthetic peptide
QSLLHSNGNNY

SEQ ID NO: 17 - skipped

SEQ ID NO: 18 - protein, 9 aa, synthetic construct
  REGION 1..9: note = Description of Artificial Sequence: Synthetic peptide
MQTLQTPLT

SEQ ID NO: 19 - protein, 8 aa, synthetic construct
  REGION 1..8: note = Description of Artificial Sequence: Synthetic peptide
GFTFSSHW

SEQ ID NO: 20 - protein, 8 aa, synthetic construct
  REGION 1..8: note = Description of Artificial Sequence: Synthetic peptide
INQDGSEK

SEQ ID NO: 21 - protein, 20 aa, synthetic construct
  REGION 1..20: note = Description of Artificial Sequence: Synthetic peptide
ARDIVLMVYDMDYYYYGMDV

SEQ ID NO: 22 - protein, 11 aa, synthetic construct
  REGION 1..11: note = Description of Artificial Sequence: Synthetic peptide
QSLHHSNGNNY

SEQ ID NO: 23 - skipped

SEQ ID NO: 24 - protein, 9 aa, synthetic construct
  REGION 1..9: note = Description of Artificial Sequence: Synthetic peptide
MQTLQTPLT

SEQ ID NO: 25 - protein, 8 aa, synthetic construct
  REGION 1..8: note = Description of Artificial Sequence: Synthetic peptide
GFTFSSHW

SEQ ID NO: 26 - protein, 8 aa, synthetic construct
  REGION 1..8: note = Description of Artificial Sequence: Synthetic peptide
INQDGSEK

SEQ ID NO: 27 - protein, 20 aa, synthetic construct
  REGION 1..20: note = Description of Artificial Sequence: Synthetic peptide
ARDIVLMVYHMDYYYYGMDV

SEQ ID NO: 28 - protein, 11 aa, synthetic construct
  REGION 1..11: note = Description of Artificial Sequence: Synthetic peptide
QSLLHSNGNNY

SEQ ID NO: 29 - skipped

SEQ ID NO: 30 - protein, 9 aa, synthetic construct
  REGION 1..9: note = Description of Artificial Sequence: Synthetic peptide
MQTLQTPLT

SEQ ID NO: 31 - protein, 8 aa, synthetic construct
  REGION 1..8: note = Description of Artificial Sequence: Synthetic peptide
GFTFSSHW

SEQ ID NO: 32 - protein, 8 aa, synthetic construct
  REGION 1..8: note = Description of Artificial Sequence: Synthetic peptide
INQDGSEK

SEQ ID NO: 33 - protein, 20 aa, synthetic construct
  REGION 1..20: note = Description of Artificial Sequence: Synthetic peptide
ARDIVLMVYHMDYYYYGMDV

SEQ ID NO: 34 - protein, 11 aa, synthetic construct
  REGION 1..11: note = Description of Artificial Sequence: Synthetic peptide
QSLHHSNGNNY

SEQ ID NO: 35 - skipped

SEQ ID NO: 36 - protein, 9 aa, synthetic construct
  REGION 1..9: note = Description of Artificial Sequence: Synthetic peptide
MQTLQTPLT

SEQ ID NO: 37 - protein, 131 aa, synthetic construct
  REGION 1..131: note = Description of Artificial Sequence: Synthetic VH; m2CX1D05 polypeptide
QVQLVQSGAEVKKPGSSVKVSCKASGGTFNSHAISWVRQAPGQGLEWMGGINPILGIANYAQKFQGRVTITADESTSTAYMELSSLRSEDTAVYYCARHYEIQIGRYGMNVYYLMYRFASWGQGTLVTVSS

SEQ ID NO: 38 - protein, 10 aa, synthetic construct
  REGION 1..10: note = Description of Artificial Sequence: Synthetic VH CDR1; m2CX1D05 peptide
GGTFNSHAIS

SEQ ID NO: 39 - protein, 20 aa, synthetic construct
  REGION 1..20: note = Description of Artificial Sequence: Synthetic VH CDR2; m2CX1D05 peptide
WMGGINPILGIANYAQKFQG

SEQ ID NO: 40 - protein, 22 aa, synthetic construct
  REGION 1..22: note = Description of Artificial Sequence: Synthetic VH CDR3; m2CX1D05 peptide
HYEIQIGRYGMNVYYLMYRFAS

SEQ ID NO: 41 - protein, 213 aa, synthetic construct
  REGION 1..213: note = Description of Artificial Sequence: Synthetic LC; m2CX1D05 polypeptide
DIQMTQSPSSLSASVGDRVTITCRASQGIRSALNWYQQKPGKAPKLLIYNGSTLQSGVPSRFSGSGSGTDFTLTISSLQPEDFAVYYCQQFDGDPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 42 - protein, 11 aa, synthetic construct
  REGION 1..11: note = Description of Artificial Sequence: Synthetic VL CDR 1; m2CX1D05 peptide
RASQGIRSALN

SEQ ID NO: 43 - protein, 11 aa, synthetic construct
  REGION 1..11: note = Description of Artificial Sequence: Synthetic VL CDR2; m2CX1D05 peptide
LLIYNGSTLQS

SEQ ID NO: 44 - protein, 7 aa, synthetic construct
  REGION 1..7: note = Description of Artificial Sequence: Synthetic VL CDR3; m2CX1D05 peptide
QQFDGDP

SEQ ID NO: 45 - protein, 119 aa, synthetic construct
  REGION 1..119: note = Description of Artificial Sequence: Synthetic VH; 1B20 polypeptide
QVQLVQSGAEVKKPGESLKISCKGSGYSFTNYWISWVRQMPGKGLEWMGIIYPGDSYTNYSPSFQGQVTISADKSISTAYLQWSSLKASDTAMYYCARDYWYKPLFDIWGQGTLVTVSS

SEQ ID NO: 46 - protein, 10 aa, synthetic construct
  REGION 1..10: note = Description of Artificial Sequence: Synthetic VH CDR1; 1B20 peptide
GYSFTNYWIS

SEQ ID NO: 47 - protein, 20 aa, synthetic construct
  REGION 1..20: note = Description of Artificial Sequence: Synthetic VH CDR2; 1B20 peptide
WMGIIYPGDSYTNYSPSFQG

SEQ ID NO: 48 - protein, 10 aa, synthetic construct
  REGION 1..10: note = Description of Artificial Sequence: Synthetic VH CDR3; 1B20 peptide
DYWYKPLFDI

SEQ ID NO: 49 - protein, 220 aa, synthetic construct
  REGION 1..220: note = Description of Artificial Sequence: Synthetic LC; 1B20 polypeptide
DIVMTQSPDSLAVSLGERATINCRSSQSVLYSSNNKNYLAWYQQKPGQPPKLLIYWASTRESGVPDRFSGSGSGTDFTLTISSLQAEDVAVYYCQQYSSFPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 50 - protein, 17 aa, synthetic construct
  REGION 1..17: note = Description of Artificial Sequence: Synthetic VL CDR1; 1B20 peptide
RSSQSVLYSSNNKNYLA

SEQ ID NO: 51 - protein, 11 aa, synthetic construct
  REGION 1..11: note = Description of Artificial Sequence: Synthetic VL CDR2; 1B20 peptide
LLIYWASTRES

SEQ ID NO: 52 - protein, 8 aa, synthetic construct
  REGION 1..8: note = Description of Artificial Sequence: Synthetic VL CDR3; 1B20 peptide
QQYSSFPI

SEQ ID NO: 53 - protein, 120 aa, synthetic construct
  REGION 1..120: note = Description of Artificial Sequence: Synthetic variable heavy antibody region polypeptide
EVQLLESGGGLVQPGGSLRLSCKASGYTFSSYGMYWVRQAPGKGLEWIGWIDPGSGGTKYNEKFKGKATISRDNSKNTLYLQMNSLRAEDTAVYYCARERYGYYFDYWGQGTLVTVSSAS

SEQ ID NO: 54 - protein, 16 aa, synthetic construct
  REGION 1..16: note = Description of Artificial Sequence: Synthetic AX132 heavy chain CDR1 antibody region peptide
KASGYTFSSYGMYWVR

SEQ ID NO: 55 - protein, 23 aa, synthetic construct
  REGION 1..23: note = Description of Artificial Sequence: Synthetic AX132 heavy chain CDR2 antibody region peptide
WIGWIDPGSGGTKYNEKFKGKAT

SEQ ID NO: 56 - protein, 15 aa, synthetic construct
  REGION 1..15: note = Description of Artificial Sequence: Synthetic AX132 heavy chain CDR3 antibody region peptide
CARERYGYYFDYWGQ

SEQ ID NO: 57 - protein, 108 aa, synthetic construct
  REGION 1..108: note = Description of Artificial Sequence: Synthetic variable light antibody region polypeptide
EIVLTQSPATLSLSPGERATITCRASQYVGSYLNWYQQKPGQAPRLLIYDASNRATGIPARFSGSGSGTDFTLTISSLEPEDFAVYYCQVWDSSPPVVFGGGTKVEIK

SEQ ID NO: 58 - protein, 17 aa, synthetic construct
  REGION 1..17: note = Description of Artificial Sequence: Synthetic AX213 and AX132 light chain CDR1 antibody region peptide
ITCRASQYVGSYLNWYQ

SEQ ID NO: 59 - protein, 13 aa, synthetic construct
  REGION 1..13: note = Description of Artificial Sequence: Synthetic AX213 and AX132 light chain CDR2 antibody region peptide
LIYDASNRATGIP

SEQ ID NO: 60 - protein, 16 aa, synthetic construct
  REGION 1..16: note = Description of Artificial Sequence: Synthetic AX132 & AX213 light chain CDR3 antibody region peptide
YYCQVWDSSPPVVFGG

SEQ ID NO: 61 - protein, 120 aa, synthetic construct
  REGION 1..120: note = Description of Artificial Sequence: Synthetic variable heavy antibody region polypeptide
EVQLLESGGGLVQPGGSLRLSCKASGYTFSRYGINWVRQAPGKGLEWIGRIDPGNGGTRYNEKFKGKATISRDNSKNTLYLQMNSLRAEDTAVYYCARANDGYSFDYWGQGTLVTVSSAS

SEQ ID NO: 62 - protein, 16 aa, synthetic construct
  REGION 1..16: note = Description of Artificial Sequence: Synthetic AX213 heavy chain CDR1 antibody region peptide
KASGYTFSRYGINWVR

SEQ ID NO: 63 - protein, 23 aa, synthetic construct
  REGION 1..23: note = Description of Artificial Sequence: Synthetic AX213 heavy chain CDR2 antibody region peptide
WIGRIDPGNGGTRYNEKFKGKAT

SEQ ID NO: 64 - protein, 15 aa, synthetic construct
  REGION 1..15: note = Description of Artificial Sequence: Synthetic AX213 heavy chain CDR3 antibody region peptide
CARANDGYSFDYWGQ

SEQ ID NO: 65 - protein, 108 aa, synthetic construct; identical to SEQ ID NO: 57
  REGION 1..108: note = Description of Artificial Sequence: Synthetic variable light antibody region polypeptide

SEQ ID NO: 66 - protein, 17 aa, synthetic construct
  REGION 1..17: note = Description of Artificial Sequence: Synthetic AX213 and AX132 light chain CDR1 antibody region peptide
ITCRASQYVGSYLNWYQ

SEQ ID NO: 67 - protein, 13 aa, synthetic construct
  REGION 1..13: note = Description of Artificial Sequence: Synthetic AX213 and AX132 light chain CDR2 antibody region peptide
LIYDASNRATGIP

SEQ ID NO: 68 - protein, 16 aa, synthetic construct
  REGION 1..16: note = Description of Artificial Sequence: Synthetic AX132 & AX213 light chain CDR3 antibody region peptide
YYCQVWDSSPPVVFGG

SEQ ID NO: 69 - protein, 119 aa, synthetic construct
  REGION 1..119: note = Description of Artificial Sequence: Synthetic AX1 VH antibody sequence polypeptide
EVQLLESGGGLVQPGGSLRLSCKASGFTFTSYYMHWVRQAPGKGLEWIGRINPDSGSTKYNEKFKGRATISRDNSKNTLYLQMNSLRAEDTAVYYCARGGRLSWDFDVWGQGTLVTVSS

SEQ ID NO: 70 - protein, 16 aa, synthetic construct
  REGION 1..16: note = Description of Artificial Sequence: Synthetic AX1 VH CDR1 antibody sequence peptide
KASGFTFTSYYMHWVR

SEQ ID NO: 71 - protein, 23 aa, synthetic construct
  REGION 1..23: note = Description of Artificial Sequence: Synthetic AX1 VH CDR2 antibody sequence peptide
WIGRINPDSGSTKYNEKFKGRAT

SEQ ID NO: 72 - protein, 16 aa, synthetic construct
  REGION 1..16: note = Description of Artificial Sequence: Synthetic AX1 VH CDR3 antibody sequence peptide
CARGGRLSWDFDVWGQ

SEQ ID NO: 73 - protein, 109 aa, synthetic construct
  REGION 1..109: note = Description of Artificial Sequence: Synthetic AX1 VL antibody sequence polypeptide
DIQMTQSPSSLSASVGDRVTITCRASQDISRYLAWYQQKPGKAPKLLIYAASSLQSGVPSRFSGSGSGTDFTLTISSLQPEDFATYYCAAYDYSLGGYVFGDGTKVEIK

SEQ ID NO: 74 - protein, 11 aa, synthetic construct
  REGION 1..11: note = Description of Artificial Sequence: Synthetic AX1 VL CDR1 antibody sequence peptide
RASQDISRYLA

SEQ ID NO: 75 - protein, 7 aa, synthetic construct
  REGION 1..7: note = Description of Artificial Sequence: Synthetic AX1 AX9 AX189 VL CDR2 antibody sequence peptide
AASSLQS

SEQ ID NO: 76 - protein, 11 aa, synthetic construct
  REGION 1..11: note = Description of Artificial Sequence: Synthetic AX1 VL CDR3 antibody sequence peptide
AAYDYSLGGYV

SEQ ID NO: 77 - protein, 121 aa, synthetic construct
  REGION 1..121: note = Description of Artificial Sequence: Synthetic AX9 AX189 VH antibody sequence polypeptide
EVQLLESGGGLVQPGGSLRLSCKASGYTFSSYWMHWVRQAPGKGLEWIGRIDPYNGGTKYNEKFKGKATISRDNSKNTLYLQMNSLRAEDTAVYYCARYGYYLGSYAMDYWGQGTLVTVSS

SEQ ID NO: 78 - protein, 16 aa, synthetic construct
  REGION 1..16: note = Description of Artificial Sequence: Synthetic AX9 AX189 VH CDR1 antibody sequence peptide
KASGYTFSSYWMHWVR

SEQ ID NO: 79 - protein, 23 aa, synthetic construct
  REGION 1..23: note = Description of Artificial Sequence: Synthetic AX9 AX189 VH CDR2 antibody sequence peptide
WIGRIDPYNGGTKYNEKFKGKAT

SEQ ID NO: 80 - protein, 18 aa, synthetic construct
  REGION 1..18: note = Description of Artificial Sequence: Synthetic AX9 AX189 VH CDR3 antibody sequence peptide
CARYGYYLGSYAMDYWGQ

SEQ ID NO: 81 - protein, 109 aa, synthetic construct
  REGION 1..109: note = Description of Artificial Sequence: Synthetic AX189 VL antibody sequence polypeptide
DIQMTQSPSSLSASVGDRVTITCRASQDVSRYLTWYQQKPGKAPKLLIYAASSLQSGVPSRFSGSGSGTDFTLTISSLQPEDFATYYCQAYDYSLSGYVFGGGTKVEIK

SEQ ID NO: 82 - protein, 11 aa, synthetic construct
  REGION 1..11: note = Description of Artificial Sequence: Synthetic AX189 VL CDR1 antibody sequence peptide
RASQDVSRYLT

SEQ ID NO: 83 - protein, 7 aa, synthetic construct
  REGION 1..7: note = Description of Artificial Sequence: Synthetic AX1 AX9 AX189 VL CDR2 antibody sequence peptide
AASSLQS

SEQ ID NO: 84 - protein, 11 aa, synthetic construct
  REGION 1..11: note = Description of Artificial Sequence: Synthetic AX189 VL CDR3 antibody sequence peptide
QAYDYSLSGYV

SEQ ID NO: 85 - protein, 115 aa, Homo sapiens
QVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSS

SEQ ID NO: 86 - protein, 5 aa, Homo sapiens
SYGIS

SEQ ID NO: 87 - protein, 17 aa, Homo sapiens
WVSFYNGNTNYAQKLQG

SEQ ID NO: 88 - protein, 6 aa, Homo sapiens
GYGMDV

SEQ ID NO: 89 - protein, 109 aa, Homo sapiens
QSALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVL

SEQ ID NO: 90 - protein, 14 aa, Homo sapiens
TGTSSDVGGYNSVS

SEQ ID NO: 91 - protein, 7 aa, Homo sapiens
EVSNRPS

SEQ ID NO: 92 - protein, 9 aa, Homo sapiens
NSYTSTSMV

SEQ ID NO: 93 - protein, 123 aa, Homo sapiens
EVQLVESGGGLVKPGGSLRLSCAASGFTFSSYSMNWVRQAPGKGLEWVSSISSSSSYISYADSVKGRFTISRDNAKNSLYLQMNSLRAEDTAVYFCARDYDFWSAYYDAFDVWGQGTMVTVSS

SEQ ID NO: 94 - protein, 10 aa, Homo sapiens
GFTFSSYSMN

SEQ ID NO: 95 - protein, 17 aa, Homo sapiens
SISSSSSYISYADSVKG

SEQ ID NO: 96 - protein, 14 aa, Homo sapiens
DYDFWSAYYDAFDV

SEQ ID NO: 97 - protein, 111 aa, Homo sapiens
QSVLTQPPSVSGAPGQRVTISCTGSSSNIGAGYDVHWYQQLPGTAPKLLISGNSNRPSGVPDRFSGSKSGTSASLAITGLQAEDEADYYCQSYDSSLSGSVFGGGTKLTVL

SEQ ID NO: 98 - protein, 14 aa, Homo sapiens
TGSSSNIGAGYDVH

SEQ ID NO: 99 - protein, 7 aa, Homo sapiens
GNSNRPS

SEQ ID NO: 100 - protein, 11 aa, Homo sapiens
QSYDSSLSGSV

SEQ ID NO: 101 - protein, 114 aa, Homo sapiens
QVQLVESGGGVAQPGRSLRLSCAASGFTFSSYGMHWVRQAPGKGLEWVAVIYYDGINKHYADSVKGRFTISRDNSKNTLYLQMNSLRAEDTAVYYCARDRGLDWGQGTLVTVSS

SEQ ID NO: 102 - protein, 10 aa, Homo sapiens
GFTFSSYGMH

SEQ ID NO: 103 - protein, 17 aa, Homo sapiens
VIYYDGINKHYADSVKG

SEQ ID NO: 104 - protein, 5 aa, Homo sapiens
DRGLD

SEQ ID NO: 105 - protein, 113 aa, Homo sapiens
DIVMTQSPDSLAVSLGERATINCKSSQSVLYSSNSKNYLVWYQQKPGQPPKLLIYWASTRESGVPDRFSGSGSGTDFTLTISSLQAEDVAVYYCQQYYSTPWTFGQGTKVEIK

SEQ ID NO: 106 - protein, 17 aa, Homo sapiens
KSSQSVLYSSNSKNYLV

SEQ ID NO: 107 - protein, 7 aa, Homo sapiens
WASTRES

SEQ ID NO: 108 - protein, 9 aa, Homo sapiens
QQYYSTPWT

SEQ ID NO: 109 - protein, 118 aa, synthetic construct
  REGION 1..118: note = Description of Artificial Sequence: Synthetic anti-PCSK9 monoclonal antibody pJG04 (clones LGT-209 and LGT-210) Vh heavy chain variable region (FR1-FR4) polypeptide
QVQLVQSGAEVKKPGASVKVSCKASGYTFSTMYMSWVRQAPGQGLEWMGRIDPANEHTNYAQKFQGRVTMTRDTSISTAYMELSRLTSDDTAVYYCARSYYYYNMDYWGQGTLVTVSS

SEQ ID NO: 110 - protein, 5 aa, synthetic construct
  REGION 1..5: note = Description of Artificial Sequence: Synthetic anti-PCSK9 monoclonal antibody clones LGT-209, LGT-210 and LGT-211 heavy chain CDR1 peptide
TMYMS

SEQ ID NO: 111 - protein, 17 aa, synthetic construct
  REGION 1..17: note = Description of Artificial Sequence: Synthetic anti-PCSK9 monoclonal antibody clones LGT-209, LGT-210 and LGT-211 heavy chain CDR2 peptide
RIDPANEHTNYAQKFQG

SEQ ID NO: 112 - protein, 9 aa, synthetic construct
  REGION 1..9: note = Description of Artificial Sequence: Synthetic anti-PCSK9 monoclonal antibody pJG04(clones LGT-209 and LGT-210) Vh heavy chain complementarity determining region 3 (CDR3) peptide
SYYYYNMDY

SEQ ID NO: 113 - protein, 106 aa, synthetic construct
  REGION 1..106: note = Description of Artificial Sequence: Synthetic anti-PCSK9 monoclonal antibody pJG10(clones LGT-209 and LGT-211) Vk light chain variable region (FR1-FR4) polypeptide
QIVLTQSPATLSVSPGERATLSCRASQSVSYMHWYQQKPGQAPRLLIYGVFRRATGIPDRFSGSGSGTDFTLTIGRLEPEDFAVYYCLQWSSDPPTFGQGTKLEIK

SEQ ID NO: 114 - protein, 10 aa, synthetic construct
  REGION 1..10: note = Description of Artificial Sequence: Synthetic anti-PCSK9 monoclonal antibody clones LGT-209, LGT-210 and LGT-211 light chain CDR1 peptide
RASQSVSYMH

SEQ ID NO: 115 - protein, 7 aa, synthetic construct
  REGION 1..7: note = Description of Artificial Sequence: Synthetic anti-PCSK9 monoclonal antibody clones LGT-209, LGT-210 and LGT-211 light chain CDR1 peptide
GVFRRAT

SEQ ID NO: 116 - protein, 9 aa, synthetic construct
  REGION 1..9: note = Description of Artificial Sequence: Synthetic mouse anti-PCSK9 monoclonal antibody LFU720 and anti-PCSK9 monoclonal antibody clones LGT-209, LGT-210 and LGT-211 light chain CDR3 peptide
LQWSSDPPT

SEQ ID NO: 117 - protein, 118 aa, Homo sapiens
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSS

SEQ ID NO: 118 - protein, 7 aa, synthetic construct
  REGION 1..7: note = Description of Artificial Sequence: Synthetic peptide
GYTFTSY

SEQ ID NO: 119 - protein, 6 aa, synthetic construct
  REGION 1..6: note = Description of Artificial Sequence: Synthetic peptide
SPFGGR

SEQ ID NO: 120 - protein, 9 aa, synthetic construct
  REGION 1..9: note = Description of Artificial Sequence: Synthetic variable heavy chain CDR peptide
ERPLYASDL

SEQ ID NO: 121 - protein, 107 aa, Homo sapiens
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIK

SEQ ID NO: 122 - protein, 11 aa, synthetic construct
  REGION 1..11: note = Description of Artificial Sequence: Synthetic variable light chain CDR peptide
RASQGISSALA

SEQ ID NO: 123 - protein, 7 aa, synthetic construct
  REGION 1..7: note = Description of Artificial Sequence: Synthetic variable light chain CDR peptide
SASYRYT

SEQ ID NO: 124 - protein, 9 aa, synthetic construct
  REGION 1..9: note = Description of Artificial Sequence: Synthetic variable light chain CDR peptide
QQRYSLWRT

SEQ ID NO: 125 - protein, 118 aa, Homo sapiens
QVQLVQSGAEVKKPGASVKVSCKASGYTFTSYYMHWVRQAPGQGLEWMGEIHPSGGRTNYNEKFKSRVTMTRDTSTSTVYMELSSLRSEDTAVYYCARERPLYAMDYWGQGTTVTVSS

SEQ ID NO: 126 - protein, 10 aa, Homo sapiens
GYTFTSYYMH

SEQ ID NO: 127 - protein, 17 aa, Homo sapiens
EIHPSGGRTNYNEKFKS

SEQ ID NO: 128 - protein, 9 aa, Homo sapiens
ERPLYAMDY

SEQ ID NO: 129 - protein, 107 aa, Homo sapiens [sequence omitted]

SEQ ID NO: 130 - protein, 11 aa, Homo sapiens
KASQDVHTAVA

SEQ ID NO: 131 - protein, 7 aa, Homo sapiens
HASYRYT

SEQ ID NO: 132 - protein, 9 aa, Homo sapiens
QQRYSLWRT

SEQ ID NO: 133 - protein, 118 aa, Homo sapiens [sequence omitted]

SEQ ID NO: 134 - protein, 10 aa, Homo sapiens
GYTFTSYYMH

SEQ ID NO: 135 - protein, 17 aa, Homo sapiens
EIHPSGGRTNYNEKFKS

SEQ ID NO: 136 - protein, 9 aa, Homo sapiens
ERPLYASDL

SEQ ID NO: 137 - protein, 107 aa, Homo sapiens [sequence omitted]

SEQ ID NO: 138 - protein, 11 aa, Homo sapiens
KASQDVHTAVA

SEQ ID NO: 139 - protein, 7 aa, Homo sapiens
HASYRYT

SEQ ID NO: 140 - protein, 9 aa, Homo sapiens
QQRYSLWRT

SEQ ID NO: 141 - protein, 118 aa, Mus musculus
QVQLQQPGAELVKPGASVKLSCKASGYTFTSYWMHWVKQRPGQGLEWIGEINPSNGRTNYNEKFKSKATLTVDKSSSTAYMQLSSLTSEDSAVYYCARERPLYAMDYWGQGTSVTVSS

SEQ ID NO: 142 - protein, 7 aa, Mus musculus
GYTFTSY

SEQ ID NO: 143 - protein, 6 aa, Mus musculus
NPSNGR

SEQ ID NO: 144 - protein, 9 aa, Mus musculus
ERPLYAMDY

SEQ ID NO: 145 - protein, 108 aa, Mus musculus
DIVMTQSHKFMSTSVGDRVSITCKASQDVSTAVAWYQQKPGQSPKLLIYSASYRYTGVPDRFTGSGSGTDFTFTISSVQAEDLAVYYCQQRYSTPRTFGGGTKLEIKR

SEQ ID NO: 146 - protein, 11 aa, Mus musculus
KASQDVSTAVA

SEQ ID NO: 147 - protein, 7 aa, synthetic construct
  REGION 1..7: note = Description of Artificial Sequence: Synthetic variable light chain CDR peptide
SASYRYT

SEQ ID NO: 148 - protein, 9 aa, Mus musculus
QQRYSTPRT

SEQ ID NO: 149 - protein, 115 aa, Mus musculus
EVQLQQSGPELVKPGASVKISCKASGYTFTDYYMNWVKQSHGKSLEWIGDINPNNGGTTYNQKFKGKATLTVDKSYSTAYMELRSLTSEDSAVYYCARWLLFAYWGQGTLVTVSA

SEQ ID NO: 150 - protein, 7 aa, Mus musculus
GYTFTDY

SEQ ID NO: 151 - protein, 6 aa, Mus musculus
NPNNGG

SEQ ID NO: 152 - protein, 6 aa, Mus musculus
WLLFAY

SEQ ID NO: 153 - protein, 108 aa, Mus musculus
DIVMTQSQKFMSTSVGDRVSVTCKASQNVGTNVAWYQQKPGQSPKALIYSASYRYSGVPDRFTGSGSGTDFTLTISNVLSEDLAEYFCQQFYSYPYTFGGGTKLEIKR

SEQ ID NO: 154 - protein, 11 aa, Mus musculus
KASQNVGTNVA

SEQ ID NO: 155 - protein, 7 aa, Mus musculus
SASYRYS

SEQ ID NO: 156 - protein, 9 aa, Mus musculus
QQFYSYPYT

SEQ ID NO: 157 - protein, 123 aa, Mus musculus
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCAGGGIYYRYDRNYFDYWGQGTTLTVSS

SEQ ID NO: 158 - protein, 7 aa, Mus musculus
GYTFTDY

SEQ ID NO: 159 - protein, 6 aa, Mus musculus
NPNNGG

SEQ ID NO: 160 - protein, 14 aa, Mus musculus
GGIYYRYDRNYFDY

SEQ ID NO: 161 - protein, 107 aa, Mus musculus
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPFTFGSGTKLEIK

SEQ ID NO: 162 - protein, 11 aa, Mus musculus
SASQGISNYLN

SEQ ID NO: 163 - protein, 7 aa, Mus musculus
YTSSLHS

SEQ ID NO: 164 - protein, 9 aa, synthetic construct
  REGION 1..9: note = Description of Artificial Sequence: Synthetic peptide
QQYSKLPFT

SEQ ID NO: 165 - protein, 117 aa, Mus musculus
EVKLVESEGGLVQPGSSMKLSCTASGFTFSDYYMAWVRQVPEKGLEWVANINYDGSNTSYLDSLKSRFIISRDNAKNILYLQMSSLKSEDTATYYCAREKFAAMDYWGQGTSVTVSS

SEQ ID NO: 166 - protein, 7 aa, Mus musculus
GFTFSDY

SEQ ID NO: 167 - protein, 6 aa, Mus musculus
NYDGSN

SEQ ID NO: 168 - protein, 8 aa, Mus musculus
EKFAAMDY

SEQ ID NO: 169 - protein, 108 aa, Mus musculus
DIVMTQSHKFMSTSFGDRVSITCKASQDVSNALAWYQQKPGHSPKLLIFSASYRYTGVPDRFTGSGSGTDFTFTISSVQAEDLAVYYCQQHYSTPWTFGGGTKLEIKR

SEQ ID NO: 170 - protein, 11 aa, Mus musculus
KASQDVSNALA

SEQ ID NO: 171 - protein, 7 aa, synthetic construct
  REGION 1..7: note = Description of Artificial Sequence: Synthetic variable light chain CDR peptide
SASYRYT

SEQ ID NO: 172 - protein, 9 aa, Mus musculus
QQHYSTPWT

SEQ ID NO: 173 - protein, 121 aa, synthetic construct
  REGION 1..121: note = Description of Artificial Sequence: Synthetic polypeptide
EVQLVESGGGLVQPGGSLRLSCAASGFTFTRHTIHWVRQAPGKGLEWVARISPANGNTNYADSVKGRFTISADTSKNTAYLQMNSLRAEDTAVYYCARWIGSRELYIMDYWGQGTLVTVSS

SEQ ID NO: 174 - protein, 10 aa, synthetic construct
  REGION 1..10: note = Description of Artificial Sequence: Synthetic peptide
GFTFTRHTIH

SEQ ID NO: 175 - protein, 17 aa, synthetic construct
  REGION 1..17: note = Description of Artificial Sequence: Synthetic peptide
RISPANGNTNYADSVKG

SEQ ID NO: 176 - protein, 12 aa, synthetic construct
  REGION 1..12: note = Description of Artificial Sequence: Synthetic peptide
WIGSRELYIMDY

SEQ ID NO: 177 - protein, 108 aa, synthetic construct
  REGION 1..108: note = Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYRIQPTFGQGTKVEIKR

SEQ ID NO: 178 - protein, 11 aa, synthetic construct
  REGION 1..11: note = Description of Artificial Sequence: Synthetic peptide
RASQDVSTAVA

SEQ ID NO: 179 - protein, 7 aa, synthetic construct
  REGION 1..7: note = Description of Artificial Sequence: Synthetic peptide
SASFLYS

SEQ ID NO: 180 - protein, 9 aa, synthetic construct
  REGION 1..9: note = Description of Artificial Sequence: Synthetic peptide
QQSYRIQPT

SEQ ID NO: 181 - protein, 121 aa, synthetic construct
  REGION 1..121: note = Description of Artificial Sequence: Synthetic polypeptide
EVQLVESGGGLVQPGGSLRLSCAASGFTFSSTAIHWVRQAPGKGLEWVARISPANGNTNYADSVKGRFTISADTSKNTAYLQMNSLRAEDTAVYYCARWIGSRELYIMDYWGQGTLVTVSS

SEQ ID NO: 182 - protein, 10 aa, synthetic construct
  REGION 1..10: note = Description of Artificial Sequence: Synthetic peptide
GFTFSSTAIH

SEQ ID NO: 183 - protein, 17 aa, synthetic construct
  REGION 1..17: note = Description of Artificial Sequence: Synthetic peptide
RISPANGNTNYADSVKG

SEQ ID NO: 184 - protein, 12 aa, synthetic construct
  REGION 1..12: note = Description of Artificial Sequence: Synthetic peptide
WIGSRELYIMDY

SEQ ID NO: 185 - protein, 108 aa, synthetic construct
  REGION 1..108: note = Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYPALHTFGQGTKVEIKR

SEQ ID NO: 186 - protein, 11 aa, synthetic construct
  REGION 1..11: note = Description of Artificial Sequence: Synthetic peptide
RASQDVSTAVA

SEQ ID NO: 187 - protein, 7 aa, synthetic construct
  REGION 1..7: note = Description of Artificial Sequence: Synthetic peptide
SASFLYS

SEQ ID NO: 188 - protein, 9 aa, synthetic construct
  REGION 1..9: note = Description of Artificial Sequence: Synthetic peptide
QQSYPALHT

SEQ ID NO: 189 - protein, 125 aa, synthetic construct
  REGION 1..125: note = Description of Artificial Sequence: Synthetic polypeptide
EVQLVESGGGLVKPGGSLRLSCAASGFPFSKLGMVWVRQAPGKGLEWVSTISSGGGYTYYPDSVKGRFTISRDNAKNSLYLQMNSLRAEDTAVYYCAREGISFQGGTYTYVMDYWGQGTLVTVSS

SEQ ID NO: 190 - protein, 10 aa, synthetic construct
  REGION 1..10: note = Description of Artificial Sequence: Synthetic peptide
GFPFSKLGMV

SEQ ID NO: 191 - protein, 17 aa, synthetic construct
  REGION 1..17: note = Description of Artificial Sequence: Synthetic peptide
TISSGGGYTYYPDSVKG

SEQ ID NO: 192 - protein, 16 aa, synthetic construct
  REGION 1..16: note = Description of Artificial Sequence: Synthetic peptide
EGISFQGGTYTYVMDY

SEQ ID NO: 193 - protein, 112 aa, synthetic construct
  REGION 1..112: note = Description of Artificial Sequence: Synthetic polypeptide
DIVMTQSPLSLPVTPGEPASISCRSSKSLLHRNGITYSYWYLQKPGQSPQLLIYQLSNLASGVPDRFSGSGSGTDFTLKISRVEAEDVGVYYCYQNLELPLTFGQGTKVEIK

SEQ ID NO: 194 - protein, 16 aa, synthetic construct
  REGION 1..16: note = Description of Artificial Sequence: Synthetic peptide
RSSKSLLHRNGITYSY

SEQ ID NO: 195 - protein, 7 aa, synthetic construct
  REGION 1..7: note = Description of Artificial Sequence: Synthetic peptide
QLSNLAS

SEQ ID NO: 196 - protein, 9 aa, synthetic construct
  REGION 1..9: note = Description of Artificial Sequence: Synthetic peptide
YQNLELPLT

SEQ ID NO: 197 - DNA, 2076 bp, Homo sapiens
  source 1..2076: mol_type = genomic DNA
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccag

SEQ ID NO: 198 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ